SEQUENCE LISTING (121 sequences)

SEQ ID NO: 1 - protein, 1556 aa, synthetic construct
  REGION 1..1556: note = fusion protein
MPKKKRKVGGSMRIFGYARVSTSQQSLDIQIRALKDAGVKANRIFTDKASGSSTDREGLDLLRMKVEEGDVILVKKLDRLSRDTADMIQLIKEFDAQGVAVRFIDDGISTDGYMGQMVVTILSAVAQAERRRILQRTNEGRQEAKLKGIKFGRRRTVDRGGSGGSGGSGGSGGSGGSMDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSRADPKKKRKV

SEQ ID NO: 2 - DNA, 4671 bp, synthetic construct
  misc_feature 1..4671: note = nucleotide sequence encoding the fusion protein of SEQ ID NO. 1
  source 1..4671: mol_type = other DNA
atgcccaaaaagaagaggaaagtaggtggcagtatgaggatatttggatacgcacgagttagtacatctcaacagtctctggacatacagataagggcattgaaggatgctggagtgaaagctaatcggatctttaccgataaggctagtggcagctctacagacagagagggcttggatctgctgagaatgaaggttgaggaaggggatgtgatactcgtcaagaaacttgacagactctcacgagacactgccgatatgatccaactgattaaggaatttgacgcccaaggtgtagccgtaagatttattgatgacggtatcagcaccgacggctatatgggccagatggtcgtcactatactgagcgctgtcgcacaagcggagagaagacgaatcctgcaacggacaaatgaaggtcgccaagaagcaaagctcaaaggaattaagttcgggcgcaggaggactgtagacagaggtggttcaggaggaagtggaggttcagggggaagtggtggctccggtgggtctatggacaagaagtactccattgggctcgccatcggcacaaacagcgtcggttgggccgtcattacggacgagtacaaggtgccgagcaaaaaattcaaagttctgggcaataccgatcgccacagcataaagaagaacctcattggcgccctcctgttcgactccggggagacggccgaagccacgcggctcaaaagaacagcacggcgcagatatacccgcagaaagaatcggatctgctacctgcaggagatctttagtaatgagatggctaaggtggatgactctttcttccataggctggaggagtcctttttggtggaggaggataaaaagcacgagcgccacccaatctttggcaatatcgtggacgaggtggcgtaccatgaaaagtacccaaccatatatcatctgaggaagaagcttgtagacagtactgataaggctgacttgcggttgatctatctcgcgctggcgcatatgatcaaatttcggggacacttcctcatcgagggggacctgaacccagacaacagcgatgtcgacaaactctttatccaactggttcagacttacaatcagcttttcgaagagaacccgatcaacgcatccggagttgacgccaaagcaatcctgagcgctaggctgtccaaatcccggcggctcgaaaacctcatcgcacagctccctggggagaagaagaacggcctgtttggtaatcttatcgccctgtcactcgggctgacccccaactttaaatctaacttcgacctggccgaagatgccaagcttcaactgagcaaagacacctacgatgatgatctcgacaatctgctggcccagatcggcgaccagtacgcagacctttttttggcggcaaagaacctgtcagacgccattctgctgagtgatattctgcgagtgaacacggagatcaccaaagctccgctgagcgctagtatgatcaagcgctatgatgagcaccaccaagacttgactttgctgaaggcccttgtcagacagcaactgcctgagaagtacaaggaaattttcttcgatcagtctaaaaatggctacgccggatacattgacggcggagcaagccaggaggaattttacaaatttattaagcccatcttggaaaaaatggacggcaccgaggagctgctggtaaagcttaacagagaagatctgttgcgcaaacagcgcactttcgacaatggaagcatcccccaccagattcacctgggcgaactgcacgctatcctcaggcggcaagaggatttctacccctttttgaaagataacagggaaaagattgagaaaatcctcacatttcggataccctactatgtaggccccctcgcccggggaaattccagattcgcgtggatgactcgcaaatcagaagagaccatcactccctggaacttcgaggaagtcgtggataagggggcctctgcccagtccttcatcgaaaggatgactaactttgataaaaatctgcctaacgaaaaggtgcttcctaaacactctctgctgtacgagtacttcacagtttataacgagctcaccaaggtcaaatacgtcacagaagggatgagaaagccagcattcctgtctggagagcagaagaaagctatcgtggacctcctcttcaagacgaaccggaaagttaccgtgaaacagctcaaagaagactatttcaaaaagattgaatgtttcgactctgttgaaatcagcggagtggaggatcgcttcaacgcatccctgggaacgtatcacgatctcctgaaaatcattaaagacaaggacttcctggacaatgaggagaacgaggacattcttgaggacattgtcctcacccttacgttgtttgaagatagggagatgattgaagaacgcttgaaaacttacgctcatctcttcgacgacaaagtcatgaaacagctcaagaggcgccgatatacaggatgggggcggctgtcaagaaaactgatcaatgggatccgagacaagcagagtggaaagacaatcctggattttcttaagtccgatggatttgccaaccggaacttcatgcagttgatccatgatgactctctcacctttaaggaggacatccagaaagcacaagtttctggccagggggacagtcttcacgagcacatcgctaatcttgcaggtagcccagctatcaaaaagggaatactgcagaccgttaaggtcgtggatgaactcgtcaaagtaatgggaaggcataagcccgagaatatcgttatcgagatggcccgagagaaccaaactacccagaagggacagaagaacagtagggaaaggatgaagaggattgaagagggtataaaagaactggggtcccaaatccttaaggaacacccagttgaaaacacccagcttcagaatgagaagctctacctgtactacctgcagaacggcagggacatgtacgtggatcaggaactggacatcaatcggctctccgactacgacgtggatgccatcgtgccccagtcttttctcaaagatgattctattgataataaagtgttgacaagatccgataaaaatagagggaagagtgataacgtcccctcagaagaagttgtcaagaaaatgaaaaattattggcggcagctgctgaacgccaaactgatcacacaacggaagttcgataatctgactaaggctgaacgaggtggcctgtctgagttggataaagccggcttcatcaaaaggcagcttgttgagacacgccagatcaccaagcacgtggcccaaattctcgattcacgcatgaacaccaagtacgatgaaaatgacaaactgattcgagaggtgaaagttattactctgaagtctaagctggtctcagatttcagaaaggactttcagttttataaggtgagagagatcaacaattaccaccatgcgcatgatgcctacctgaatgcagtggtaggcactgcacttatcaaaaaatatcccaagcttgaatctgaatttgtttacggagactataaagtgtacgatgttaggaaaatgatcgcaaagtctgagcaggaaataggcaaggccaccgctaagtacttcttttacagcaatattatgaattttttcaagaccgagattacactggccaatggagagattcggaagcgaccacttatcgaaacaaacggagaaacaggagaaatcgtgtgggacaagggtagggatttcgcgacagtccggaaggtcctgtccatgccgcaggtgaacatcgttaaaaagaccgaagtacagaccggaggcttctccaaggaaagtatcctcccgaaaaggaacagcgacaagctgatcgcacgcaaaaaagattgggaccccaagaaatacggcggattcgattctcctacagtcgcttacagtgtactggttgtggccaaagtggagaaagggaagtctaaaaaactcaaaagcgtcaaggaactgctgggcatcacaatcatggagcgatcaagcttcgaaaaaaaccccatcgactttctcgaggcgaaaggatataaagaggtcaaaaaagacctcatcattaagcttcccaagtactctctctttgagcttgaaaacggccggaaacgaatgctcgctagtgcgggcgagctgcagaaaggtaacgagctggcactgccctctaaatacgttaatttcttgtatctggccagccactatgaaaagctcaaagggtctcccgaagataatgagcagaagcagctgttcgtggaacaacacaaacactaccttgatgagatcatcgagcaaataagcgaattctccaaaagagtgatcctcgccgacgctaacctcgataaggtgctttctgcttacaataagcacagggataagcccatcagggagcaggcagaaaacattatccacttgtttactctgaccaacttgggcgcgcctgcagccttcaagtacttcgacaccaccatagacagaaagcggtacacctctacaaaggaggtcctggacgccacactgattcatcagtcaattacggggctctatgaaacaagaatcgacctctctcagctcggtggagacagcagggctgaccccaagaagaagaggaaggtgtga

SEQ ID NO: 3 - DNA, 18 bp, synthetic construct
  misc_feature 1..18: note = sgRNA guide sequence
  source 1..18: mol_type = other DNA
agaagagcgagctcttct

SEQ ID NO: 4 - DNA, 20 bp, synthetic construct
  misc_feature 1..20: note = sgRNA guide sequence
  source 1..20: mol_type = other DNA
cgaacgtacgagtgcaagcc

SEQ ID NO: 5 - DNA, 20 bp, synthetic construct
  misc_feature 1..20: note = sgRNA guide sequence
  source 1..20: mol_type = other DNA
gaacgtacgagtgcaagcct

SEQ ID NO: 6 - DNA, 20 bp, synthetic construct
  misc_feature 1..20: note = sgRNA guide sequence
  source 1..20: mol_type = other DNA
aacgtacgagtgcaagcctg

SEQ ID NO: 7 - DNA, 20 bp, synthetic construct
  misc_feature 1..20: note = sgRNA guide sequence
  source 1..20: mol_type = other DNA
acgtacgagtgcaagcctgg

SEQ ID NO: 8 - DNA, 20 bp, synthetic construct
  misc_feature 1..20: note = sgRNA guide sequence
  source 1..20: mol_type = other DNA
acgagtgcaagcctggggga

SEQ ID NO: 9 - DNA, 20 bp, synthetic construct
  misc_feature 1..20: note = sgRNA guide sequence
  source 1..20: mol_type = other DNA
tgcaagcctgggggatggat

SEQ ID NO: 10 - DNA, 20 bp, synthetic construct
  misc_feature 1..20: note = sgRNA guide sequence
  source 1..20: mol_type = other DNA
cagacagaccatactccaga

SEQ ID NO: 11 - DNA, 20 bp, synthetic construct
  misc_feature 1..20: note = sgRNA guide sequence
  source 1..20: mol_type = other DNA
agacagaccatactccagat

SEQ ID NO: 12 - DNA, 20 bp, synthetic construct
  misc_feature 1..20: note = sgRNA guide sequence
  source 1..20: mol_type = other DNA
gacagaccatactccagatg

SEQ ID NO: 13 - DNA, 20 bp, synthetic construct
  misc_feature 1..20: note = sgRNA guide sequence
  source 1..20: mol_type = other DNA
acagaccatactccagatgg

SEQ ID NO: 14 - DNA, 20 bp, synthetic construct
  misc_feature 1..20: note = sgRNA guide sequence
  source 1..20: mol_type = other DNA
accatactccagatggggga

SEQ ID NO: 15 - DNA, 20 bp, synthetic construct
  misc_feature 1..20: note = sgRNA guide sequence
  source 1..20: mol_type = other DNA
actccagatgggggatggct

SEQ ID NO: 16 - DNA, 18 bp, synthetic construct
  misc_feature 1..18: note = sgRNA guide sequence
  source 1..18: mol_type = other DNA
gggtcttcgagaagacct

SEQ ID NO: 17 - DNA, 21 bp, synthetic construct
  misc_feature 1..21: note = sgRNA guide sequence
  source 1..21: mol_type = other DNA
gacgtacgagtgcaagcctgg

SEQ ID NO: 18 - DNA, 21 bp, synthetic construct
  misc_feature 1..21: note = sgRNA guide sequence
  source 1..21: mol_type = other DNA
gacagaccttactccagaagg

SEQ ID NO: 19 - DNA, 21 bp, synthetic construct
  misc_feature 1..21: note = sgRNA guide sequence
  source 1..21: mol_type = other DNA
gtgcaagcctgggggaaggat

SEQ ID NO: 20 - DNA, 21 bp, synthetic construct
  misc_feature 1..21: note = sgRNA guide sequence
  source 1..21: mol_type = other DNA
gactccagaagggggaaggct

SEQ ID NO: 21 - DNA, 21 bp, synthetic construct
  misc_feature 1..21: note = sgRNA guide sequence
  source 1..21: mol_type = other DNA
gacgagtgcaagcctggggga

SEQ ID NO: 22 - DNA, 21 bp, synthetic construct
  misc_feature 1..21: note = sgRNA guide sequence
  source 1..21: mol_type = other DNA
gaccttactccagaaggggga

SEQ ID NO: 23 - DNA, 21 bp, synthetic construct
  misc_feature 1..21: note = sgRNA guide sequence
  source 1..21: mol_type = other DNA
gttgctcaccatggtggcgac

SEQ ID NO: 24 - DNA, 21 bp, synthetic construct
  misc_feature 1..21: note = primer
  source 1..21: mol_type = other DNA
gctcgcccttgctcaccatgg

SEQ ID NO: 25 - DNA, 21 bp, synthetic construct
  misc_feature 1..21: note = sgRNA guide sequence
  source 1..21: mol_type = other DNA
gctcctcgcccttgctcacca

SEQ ID NO: 26 - DNA, 20 bp, synthetic construct
  misc_feature 1..20: note = sgRNA guide sequence
  source 1..20: mol_type = other DNA
ggtcgccaccatggtgagca

SEQ ID NO: 27 - DNA, 20 bp, synthetic construct
  misc_feature 1..20: note = sgRNA guide sequence
  source 1..20: mol_type = other DNA
gtcgccaccatggtgagcaa

SEQ ID NO: 28 - DNA, 21 bp, synthetic construct
  misc_feature 1..21: note = sgRNA guide sequence
  source 1..21: mol_type = other DNA
gcaccatggtgagcaagggcg

SEQ ID NO: 29 - DNA, 20 bp, synthetic construct
  misc_feature 1..20: note = primer
  source 1..20: mol_type = other DNA
cgcatatgtggtgttgaaga

SEQ ID NO: 30 - DNA, 22 bp, synthetic construct
  misc_feature 1..22: note = primer
  source 1..22: mol_type = other DNA
ctagggctttctgctctgtcat

SEQ ID NO: 31 - DNA, 20 bp, synthetic construct
  misc_feature 1..20: note = primer
  source 1..20: mol_type = other DNA
tggagggcacagttaagccg

SEQ ID NO: 32 - DNA, 20 bp, synthetic construct
  misc_feature 1..20: note = primer
  source 1..20: mol_type = other DNA
aataccgcctttgagtgagc

SEQ ID NO: 33 - DNA, 20 bp, synthetic construct
  misc_feature 1..20: note = primer
  source 1..20: mol_type = other DNA
agctgtgaccggcgcctacg

SEQ ID NO: 34 - DNA, 23 bp, synthetic construct
  misc_feature 1..23: note = primer
  source 1..23: mol_type = other DNA
ctgagcacccagtccgccctgag

SEQ ID NO: 35 - DNA, 47 bp, synthetic construct
  misc_feature 1..47: note = primer
  source 1..47: mol_type = other DNA
aattctccgatccatcccccaggcttgcactcgtacgttcgaaatat

SEQ ID NO: 36 - DNA, 46 bp, synthetic construct
  misc_feature 1..46: note = primer
  source 1..46: mol_type = other DNA
ataatatttcgaacgtacgagtgcaagcctgggggatggatcggag

SEQ ID NO: 37 - DNA, 47 bp, synthetic construct
  misc_feature 1..47: note = primer
  source 1..47: mol_type = other DNA
tataaattatcagacagaccatactccagatgggggatggctaggtg

SEQ ID NO: 38 - DNA, 48 bp, synthetic construct
  misc_feature 1..48: note = primer
  source 1..48: mol_type = other DNA
aattcacctagccatcccccatctggagtatggtctgtctgataattt

SEQ ID NO: 39 - DNA, 47 bp, synthetic construct
  misc_feature 1..47: note = primer
  source 1..47: mol_type = other DNA
cgcgttccgatccatcccccaggcttgcactcgtacgttcgaaatat

SEQ ID NO: 40 - DNA, 46 bp, synthetic construct
  misc_feature 1..46: note = primer
  source 1..46: mol_type = other DNA
ataatatttcgaacgtacgagtgcaagcctgggggatggatcggaa

SEQ ID NO: 41 - DNA, 47 bp, synthetic construct
  misc_feature 1..47: note = primer
  source 1..47: mol_type = other DNA
tataaattatcagacagaccatactccagatgggggatggctaggta

SEQ ID NO: 42 - DNA, 48 bp, synthetic construct
  misc_feature 1..48: note = primer
  source 1..48: mol_type = other DNA
cgcgtacctagccatcccccatctggagtatggtctgtctgataattt

SEQ ID NO: 43 - DNA, 47 bp, synthetic construct
  misc_feature 1..47: note = primer
  source 1..47: mol_type = other DNA
aattctccgatccttcccccaggcttgcactcgtacgttcgaaatat

SEQ ID NO: 44 - DNA, 43 bp, synthetic construct
  misc_feature 1..43: note = primer
  source 1..43: mol_type = other DNA
ctccgatccttcccccaggcttgcactcgtacgttcgaaatat

SEQ ID NO: 45 - DNA, 46 bp, synthetic construct
  misc_feature 1..46: note = primer
  source 1..46: mol_type = other DNA
ataatatttcgaacgtacgagtgcaagcctgggggaaggatcggag

SEQ ID NO: 46 - DNA, 47 bp, synthetic construct
  misc_feature 1..47: note = primer
  source 1..47: mol_type = other DNA
tataaattatcagacagaccttactccagaagggggaaggctaggtg

SEQ ID NO: 47 - DNA, 48 bp, synthetic construct
  misc_feature 1..48: note = primer
  source 1..48: mol_type = other DNA
gatccacctagccttcccccttctggagtaaggtctgtctgataattt

SEQ ID NO: 48 - DNA, 44 bp, synthetic construct
  misc_feature 1..44: note = primer
  source 1..44: mol_type = other DNA
cacctagccttcccccttctggagtaaggtctgtctgataattt

SEQ ID NO: 49 - DNA, 42 bp, synthetic construct
  misc_feature 1..42: note = primer
  source 1..42: mol_type = other DNA
agaacagttgatagaggagggagcgggggaagcggtggctca

SEQ ID NO: 50 - DNA, 42 bp, synthetic construct
  misc_feature 1..42: note = primer
  source 1..42: mol_type = other DNA
cattgagccaccgcttcccccgctccctcctctatcaactgt

SEQ ID NO: 51 - DNA, 36 bp, synthetic construct
  misc_feature 1..36: note = primer
  source 1..36: mol_type = other DNA
agaactgttgaccgaggtggttcaggaggaagtgga

SEQ ID NO: 52 - DNA, 36 bp, synthetic construct
  misc_feature 1..36: note = primer
  source 1..36: mol_type = other DNA
acctccacttcctcctgaaccacctcggtcaacagt

SEQ ID NO: 53 - DNA, 33 bp, synthetic construct
  misc_feature 1..33: note = primer
  source 1..33: mol_type = other DNA
ggttcagggggaagtggtggctccggtgggtct

SEQ ID NO: 54 - DNA, 33 bp, synthetic construct
  misc_feature 1..33: note = primer
  source 1..33: mol_type = other DNA
catagacccaccggagccaccacttccccctga

SEQ ID NO: 55 - DNA, 30 bp, synthetic construct
  misc_feature 1..30: note = primer
  source 1..30: mol_type = other DNA
agaacagttgatcggagcggttctgagact

SEQ ID NO: 56 - DNA, 30 bp, synthetic construct
  misc_feature 1..30: note = primer
  source 1..30: mol_type = other DNA
cggagtctcagaaccgctccgatcaactgt

SEQ ID NO: 57 - DNA, 33 bp, synthetic construct
  misc_feature 1..33: note = primer
  source 1..33: mol_type = other DNA
ccgggaacctcagagtctgctacgccggaaagc

SEQ ID NO: 58 - DNA, 33 bp, synthetic construct
  misc_feature 1..33: note = primer
  source 1..33: mol_type = other DNA
catgctttccggcgtagcagactctgaggttcc

SEQ ID NO: 59 - DNA, 37 bp, synthetic construct
  misc_feature 1..37: note = primer
  source 1..37: mol_type = other DNA
agaaccgtagatcgcgggggctctggaggatcaggta

SEQ ID NO: 60 - DNA, 37 bp, synthetic construct
  misc_feature 1..37: note = primer
  source 1..37: mol_type = other DNA
cgctacctgatcctccagagcccccgcgatctacggt

SEQ ID NO: 61 - DNA, 41 bp, synthetic construct
  misc_feature 1..41: note = primer
  source 1..41: mol_type = other DNA
gcgaaacgccgggtactagcgaaagcgcgacacctgagagt

SEQ ID NO: 62 - DNA, 41 bp, synthetic construct
  misc_feature 1..41: note = primer
  source 1..41: mol_type = other DNA
catactctcaggtgtcgcgctttcgctagtacccggcgttt

SEQ ID NO: 63 - DNA, 34 bp, synthetic construct
  misc_feature 1..34: note = primer
  source 1..34: mol_type = other DNA
acggctcttcgatgcccaaaaagaagaggaaagt

SEQ ID NO: 64 - DNA, 32 bp, synthetic construct
  misc_feature 1..32: note = primer
  source 1..32: mol_type = other DNA
agcgctcttcatctgtctacagtcctcctgcg

SEQ ID NO: 65 - DNA, 32 bp, synthetic construct
  misc_feature 1..32: note = primer
  source 1..32: mol_type = other DNA
acggctcttcgcatttttttcccgggggatcc

SEQ ID NO: 66 - DNA, 29 bp, synthetic construct
  misc_feature 1..29: note = primer
  source 1..29: mol_type = other DNA
gcgctcttcagccatcggcacaaacagcg

SEQ ID NO: 67 - DNA, 35 bp, synthetic construct
  misc_feature 1..35: note = primer
  source 1..35: mol_type = other DNA
acggctcttcgggcgagcccaatggagtacttctt

SEQ ID NO: 68 - DNA, 31 bp, synthetic construct
  misc_feature 1..31: note = primer
  source 1..31: mol_type = other DNA
gcgctcttcagccatcgtgccccagtctttt

SEQ ID NO: 69 - DNA, 33 bp, synthetic construct
  misc_feature 1..33: note = primer
  source 1..33: mol_type = other DNA
acggctcttcgggcatccacgtcgtagtcggag

SEQ ID NO: 70 - DNA, 39 bp, synthetic construct
  misc_feature 1..39: note = primer
  source 1..39: mol_type = other DNA
atacaccggtgccaccatgcccaaaaagaagaggaaagt

SEQ ID NO: 71 - DNA, 33 bp, synthetic construct
  misc_feature 1..33: note = primer
  source 1..33: mol_type = other DNA
gatgacaattgtcacaccttcctcttcttcttg

SEQ ID NO: 72 - DNA, 38 bp, synthetic construct
  misc_feature 1..38: note = primer
  source 1..38: mol_type = other DNA
gtgagaattctctttgaaaagataatgtatgattatgc

SEQ ID NO: 73 - DNA, 39 bp, synthetic construct
  misc_feature 1..39: note = primer
  source 1..39: mol_type = other DNA
acggctcttcgtctttgaaaagataatgtatgattatgc

SEQ ID NO: 74 - DNA, 43 bp, synthetic construct
  misc_feature 1..43: note = primer
  source 1..43: mol_type = other DNA
acggctcttcgagagtctccaattatctagtaaaaaaagcacc

SEQ ID NO: 75 - DNA, 47 bp, synthetic construct
  misc_feature 1..47: note = primer
  source 1..47: mol_type = other DNA
cgtcatgtcactagtagagtctccaattatctagtaaaaaaagcacc

SEQ ID NO: 76 - DNA, 30 bp, synthetic construct
  misc_feature 1..30: note = primer
  source 1..30: mol_type = other DNA
gtgagaattcgagggcctatttcccatgat

SEQ ID NO: 77 - DNA, 30 bp, synthetic construct
  misc_feature 1..30: note = primer
  source 1..30: mol_type = other DNA
acggctcttcgtctgtctgcagaattggcg

SEQ ID NO: 78 - DNA, 34 bp, synthetic construct
  misc_feature 1..34: note = primer
  source 1..34: mol_type = other DNA
agcgctcttctagagagggcctatttcccatgat

SEQ ID NO: 79 - DNA, 34 bp, synthetic construct
  misc_feature 1..34: note = primer
  source 1..34: mol_type = other DNA
cgtcatgtctctagatttgtctgcagaattggcg

SEQ ID NO: 80 - DNA, 23 bp, synthetic construct
  misc_feature 1..23: note = primer
  source 1..23: mol_type = other DNA
atccgaacgtacgagtgcaagcc

SEQ ID NO: 81 - DNA, 23 bp, synthetic construct
  misc_feature 1..23: note = primer
  source 1..23: mol_type = other DNA
aacggcttgcactcgtacgttcg

SEQ ID NO: 82 - DNA, 23 bp, synthetic construct
  misc_feature 1..23: note = primer
  source 1..23: mol_type = other DNA
atcgaacgtacgagtgcaagcct

SEQ ID NO: 83 - DNA, 23 bp, synthetic construct
  misc_feature 1..23: note = primer
  source 1..23: mol_type = other DNA
aacaggcttgcactcgtacgttc

SEQ ID NO: 84 - DNA, 23 bp, synthetic construct
  misc_feature 1..23: note = primer
  source 1..23: mol_type = other DNA
atcaacgtacgagtgcaagcctg

SEQ ID NO: 85 - DNA, 23 bp, synthetic construct
  misc_feature 1..23: note = primer
  source 1..23: mol_type = other DNA
aaccaggcttgcactcgtacgtt

SEQ ID NO: 86 - DNA, 23 bp, synthetic construct
  misc_feature 1..23: note = primer
  source 1..23: mol_type = other DNA
atcacgtacgagtgcaagcctgg

SEQ ID NO: 87 - DNA, 23 bp, synthetic construct
  misc_feature 1..23: note = primer
  source 1..23: mol_type = other DNA
aacccaggcttgcactcgtacgt

SEQ ID NO: 88 - DNA, 23 bp, synthetic construct
  misc_feature 1..23: note = primer
  source 1..23: mol_type = other DNA
atcacgagtgcaagcctggggga

SEQ ID NO: 89 - DNA, 23 bp, synthetic construct
  misc_feature 1..23: note = primer
  source 1..23: mol_type = other DNA
aactcccccaggcttgcactcgt

SEQ ID NO: 90 - DNA, 23 bp, synthetic construct
  misc_feature 1..23: note = primer
  source 1..23: mol_type = other DNA
atctgcaagcctgggggatggat

SEQ ID NO: 91 - DNA, 23 bp, synthetic construct
  misc_feature 1..23: note = primer
  source 1..23: mol_type = other DNA
aacatccatcccccaggcttgca

SEQ ID NO: 92 - DNA, 23 bp, synthetic construct
  misc_feature 1..23: note = primer
  source 1..23: mol_type = other DNA
atccagacagaccatactccaga

SEQ ID NO: 93 - DNA, 23 bp, synthetic construct
  misc_feature 1..23: note = primer
  source 1..23: mol_type = other DNA
aactctggagtatggtctgtctg

SEQ ID NO: 94 - DNA, 23 bp, synthetic construct
  misc_feature 1..23: note = primer
  source 1..23: mol_type = other DNA
atcagacagaccatactccagat

SEQ ID NO: 95 - DNA, 23 bp, synthetic construct
  misc_feature 1..23: note = primer
  source 1..23: mol_type = other DNA
aacatctggagtatggtctgtct

SEQ ID NO: 96 - DNA, 23 bp, synthetic construct
  misc_feature 1..23: note = primer
  source 1..23: mol_type = other DNA
atcgacagaccatactccagatg

SEQ ID NO: 97 - DNA, 23 bp, synthetic construct
  misc_feature 1..23: note = primer
  source 1..23: mol_type = other DNA
aaccatctggagtatggtctgtc

SEQ ID NO: 98 - DNA, 23 bp, synthetic construct
  misc_feature 1..23: note = primer
  source 1..23: mol_type = other DNA
atcacagaccatactccagatgg

SEQ ID NO: 99 - DNA, 23 bp, synthetic construct
  misc_feature 1..23: note = primer
  source 1..23: mol_type = other DNA
aacccatctggagtatggtctgt

SEQ ID NO: 100 - DNA, 23 bp, synthetic construct
  misc_feature 1..23: note = primer
  source 1..23: mol_type = other DNA
atcaccatactccagatggggga

SEQ ID NO: 101 - DNA, 23 bp, synthetic construct
  misc_feature 1..23: note = primer
  source 1..23: mol_type = other DNA
aactcccccatctggagtatggt

SEQ ID NO: 102 - DNA, 23 bp, synthetic construct
  misc_feature 1..23: note = primer
  source 1..23: mol_type = other DNA
atcactccagatgggggatggct

SEQ ID NO: 103 - DNA, 23 bp, synthetic construct
  misc_feature 1..23: note = primer
  source 1..23: mol_type = other DNA
aacagccatcccccatctggagt

SEQ ID NO: 104 - DNA, 25 bp, synthetic construct
  misc_feature 1..25: note = primer
  source 1..25: mol_type = other DNA
caccgacgtacgagtgcaagcctgg

SEQ ID NO: 105 - DNA, 25 bp, synthetic construct
  misc_feature 1..25: note = primer
  source 1..25: mol_type = other DNA
aaacccaggcttgcactcgtacgtc

SEQ ID NO: 106 - DNA, 25 bp, synthetic construct
  misc_feature 1..25: note = primer
  source 1..25: mol_type = other DNA
caccgacagaccttactccagaagg

SEQ ID NO: 107 - DNA, 25 bp, synthetic construct
  misc_feature 1..25: note = primer
  source 1..25: mol_type = other DNA
aaacccttctggagtaaggtctgtc

SEQ ID NO: 108 - DNA, 25 bp, synthetic construct
  misc_feature 1..25: note = primer
  source 1..25: mol_type = other DNA
caccgtgcaagcctgggggaaggat

SEQ ID NO: 109 - DNA, 25 bp, synthetic construct
  misc_feature 1..25: note = primer
  source 1..25: mol_type = other DNA
aaacatccttcccccaggcttgcac

SEQ ID NO: 110 - DNA, 25 bp, synthetic construct
  misc_feature 1..25: note = primer
  source 1..25: mol_type = other DNA
caccgactccagaagggggaaggct

SEQ ID NO: 111 - DNA, 25 bp, synthetic construct
  misc_feature 1..25: note = primer
  source 1..25: mol_type = other DNA
aaacagccttcccccttctggagtc

SEQ ID NO: 112 - DNA, 25 bp, synthetic construct
  misc_feature 1..25: note = primer
  source 1..25: mol_type = other DNA
caccgacgagtgcaagcctggggga

SEQ ID NO: 113 - DNA, 25 bp, synthetic construct
  misc_feature 1..25: note = primer
  source 1..25: mol_type = other DNA
aaactcccccaggcttgcactcgtc

SEQ ID NO: 114 - DNA, 25 bp, synthetic construct
  misc_feature 1..25: note = primer
  source 1..25: mol_type = other DNA
caccgaccttactccagaaggggga

SEQ ID NO: 115 - DNA, 25 bp, synthetic construct
  misc_feature 1..25: note = primer
  source 1..25: mol_type = other DNA
aaactcccccttctggagtaaggtc

SEQ ID NO: 116 - DNA, 88 bp, synthetic construct
  misc_feature 1..88: note = iCas9 site
  source 1..88: mol_type = other DNA
tccgatccatcccccaggcttgcactcgtacgttcgaaatattataaattatcagacagaccatactccagatgggggatggctaggt

SEQ ID NO: 117 - DNA, 88 bp, synthetic construct
  misc_feature 1..88: note = iCas9 site
  source 1..88: mol_type = other DNA
tccgatccttcccccaggcttgcactcgtacgttcgaaatattataaattatcagacagaccttactccagaagggggaaggctaggt

SEQ ID NO: 118 - DNA, 123 bp, synthetic construct
  misc_feature 1..123: note = iCas9 site with accessory site
  source 1..123: mol_type = other DNA
tccgatccttcccccaggcttgcactcgtacgttcgaaatattataaattatcagacagaccttactccagaagggggaaggctaggtggctaccggtcgccaccatggtgagcaagggcgag

SEQ ID NO: 119 - DNA, 123 bp, synthetic construct; identical to SEQ ID NO: 118
  misc_feature 1..123: note = iCas9 site with accessory site
  source 1..123: mol_type = other DNA

SEQ ID NO: 120 - protein, 16 aa, synthetic construct
  REGION 1..16: note = linker
SGSETPGTSESATPES

SEQ ID NO: 121 - protein, 21 aa, synthetic construct
  REGION 1..21: note = linker
GGSGGSGSETPGTSESATPES

(19) United States
(12) Patent Application Publication
(10) Pub. No.: US 2026/0218243 A1
(43) Pub. Date: Jul. 30, 2026

(54) CAS9 FUSION PROTEINS AND RELATED METHODS

(71) Applicant: Arizona Board of Regents on Behalf of Arizona State University, Scottsdale, AZ (US)

(72) Inventors: Kylie STANDAGE-BEIER, Phoenix, AZ (US); Parithi BALACHANDRAN, Coimbatore (IN); Nicholas BROOKHOUSER, Tempe, AZ (US); David BRAFMAN, Phoenix, AZ (US); Xiao WANG, Chandler, AZ (US)

(21) Appl. No.: 19/646,414

(22) Filed: Apr. 13, 2026

Related U.S. Application Data
(63) Continuation of application No. 17/602,581, filed on Apr. 14, 2020.
(60) Provisional application No. 62/834,880, filed on Apr. 16, 2019.

(51) Int. Cl. C12N 15/90; C12N 9/16; C12N 9/22; C12N 15/11

(52) U.S. Cl. CPC C12N 15/907; C12N 9/16; C12N 9/22; C12N 15/11; C12Y 301/21; C12N 2310/20; C12N 2800/80

(57) ABSTRACT

Disclosed are recombinant Cas9 proteins, methods of production, and methods of use for targeted DNA deletions, DNA insertions, or both in a eukaryotic genome. An assay system for evaluating the ability of the recombinant Cas9 proteins for targeted DNA deletions, DNA insertions, or both in a eukaryotic genome is also disclosed.

### RELATED APPLICATIONS

This application is a divisional of U.S. Utility application Ser. No. 17/602,581, filed Oct. 8, 2021, which is the U.S. National Stage of International patent application no. PCT/US2020/028149, filed Apr. 14, 2020, which claims the benefit of U.S. provisional patent application No. 62/834,880, filed Apr. 16, 2019 titled “CAS9 Fusion Proteins and Related Methods,” the contents of each of which are hereby incorporated by reference in their entireties.

### STATEMENT REGARDING FEDERALLY SPONSORED RESEARCH OR DEVELOPMENT

This invention was made with government support under GM106081 awarded by the National Institutes of Health. The government has certain rights in the invention.

### INCORPORATION-BY-REFERENCE OF MATERIAL ELECTRONICALLY FILED

Incorporated by reference in its entirety herein is a computer-readable nucleotide/amino acid sequence listing submitted concurrently herewith and identified as follows: One 160,438 byte XML file named “20260413_SeqList.xml” created on Apr. 13, 2026.

### TECHNICAL FIELD

The disclosure is directed to recombinant Cas9 fusion proteins capable of targeted DNA deletion and DNA integration in a cell without triggering the cell's endogenous DNA repair mechanism such as, homologous recombination. The Cas9 fusion proteins disclosed herein also minimize off target mutations, nucleotide insertions, and/or nucleotide deletions.

### BACKGROUND

Clustered regularly interspaced short palindromic repeats (CRISPR) and CRISPR-associated (Cas) systems, such as Cas9 nuclease and Cas12a (Cpf1), have drastically improved the ease of targeted DNA modifications, largely due to its ability to target Cas9's function via design and co-expression of single guide RNAs (sgRNAs) or CRISPR RNA (crRNAs) for Cas12a. In the case of Cas9, sgRNA targeting is straightforward as it requires only simple DNA-RNA base pairing combined with the presence of a protospacer adjacent motif (PAM) on the target DNA. Systems employing Cas9 are highly robust and function in a broad range of organisms for a variety of editing strategies. Strategies for DNA integration and deletion are largely accomplished via formation of DSBs or paired single-stranded DNA breaks (SSBs) followed by processing via endogenous non-homologous end joining (NHEJ) or homologous recombination (HR). More recently, groups have described homology independent target integration (HITI), an effective technique for NHEJ mediated genome integration. This technique produces simultaneous CRISPR-Cas9-targeted double-stranded breaks (DSBs) on plasmid and genomic protospacer sequences and then utilize NHEJ to ligate plasmid DNA into the genomic protospacer. However, it has become apparent that CRISPR-based genome engineering strategies are limited with respect to their dependence on the generation of DSBs and endogenous DNA repair machinery. DSBs could generate unwanted mutations, translocations, complex rearrangements and destabilize karyotype. This is a fundamental limitation of CRISPR-Cas9's application in editing human cell lines for basic science and therapeutic purposes.

Technologies that avoid incurring double-stranded DNA damages during the editing process include “base-editor” (BE) Cas9 systems, which enable generation of single nucleotide changes without the need for double stranded DNA breaks. BE-Cas9's accomplished single nucleotide changes via fusion of a nicking Cas9 (Cas9D10A) with a cytidine deaminase and uracil glycosylase inhibitor domains. However, BEs are limited to single nucleotide changes. Accordingly, additional developments in the CRISPR-Cas9 technology is needed to prevent the development of unwanted mutations, translocations, complex rearrangements and destabilized karyotype.

### SUMMARY

The disclosure is directed to a recombinant Cas9. The recombinant Cas9 preferably comprises a catalytic domain of the resolvase of transposon Tn3 (“Tn3 resolvase”). In some aspects, the disclosure is directed to a Cas9 fusion protein where a catalytically inactive Cas9 is fused with the catalytic domain of a hyperactive mutant Tn3 resolvase. In certain nonlimiting embodiments, the catalytically inactive Cas9 is dCas9. A recombinant Cas9 comprising dCas9 and the catalytic domain of a hyperactive mutant Tn3 resolvase is referred to herein as iCas9.

In some aspects, the dimer of the recombinant Cas9 is described, wherein the dimer is bound to a DNA molecule. In certain embodiments of the dimer, the recombinant Cas9 further comprises a single guide RNA (sgRNA) bound to the catalytically inactive Cas9, and the DNA molecule on which the dimer is bound comprises two binding sites for the sgRNA. The distance between the binding sites for the sgRNA is at least 21 bp, for example, at least 22 bp, 22 bp, 30 bp, 31 bp, 40 bp, or 44 bp. In certain embodiments, the fusion protein of the dimer is bound to the same strand of the DNA molecule. In other embodiments, the fusion protein of the dimer is bound to opposite strands of the DNA molecule.

In some aspects, the tetramer of the recombinant Cas9 is described, wherein the tetramer is bound to a DNA molecule. In some embodiments of the tetramer, the recombinant Cas9 further comprises a sgRNA bound to the catalytically inactive Cas9, and the DNA molecule on which the tetramer is bound comprises two binding sites for the sgRNA. The distance between the binding sites for the sgRNA is at least 21 bp, for example, at least 22 bp, 22 bp, 30 bp, 31 bp, 40 bp, or 44 bp. In certain embodiments, each dimer of the tetramer is bound to the same strand of the DNA molecule. In other embodiments, each dimer of the tetramer is bound to opposite strands of the DNA molecule.

The disclosure is also directed to a method of producing the recombinant Cas9 and the use of the recombinant Cas9 for targeted DNA deletion or targeted DNA insertion in an eukaryotic genome. Kits for evaluating the ability of the recombination Cas9 for targeted DNA deletion or targeted DNA insertion in an eukaryotic genome are also disclosed herein.

### BRIEF DESCRIPTION OF THE DRAWINGS

FIGS. 1A-1C depict the design of iCas9 and iCas9 target sites. In accordance with certain embodiments, FIG. 1A shows the architecture of the iCas9 fusion protein. A Catalytically inactive Cas9 (dCas9) is fused to the catalytic domain of a hyperactive mutant recombinase from transposon TN3 (mTN3). dCas9 and mTN3 are separated by a flexible linker region (GGS*6). To promote nuclear entry, both N- and C-Termini have SV40 nuclear localization signals sequences (NLS). Given catalytic domains are fused to dCas9, iCas9 is guided via single guide RNAs (sgRNAs). In accordance with certain embodiments, FIG. 1B shows mTN3 function is dependent on dimerization on target site sequences followed by tetramerization. Tetramerization results in recombination, which can occur in two directions: deletion or integration. iCas9 can target either DNA deletion if target recognition sites are located on the same molecule (left), or alternatively iCas9 can target DNA integration if target sites are on separate DNAs (right). FIG. 1C depicts, in accordance with certain embodiments, the design of an iCas9 recognition site consists of two sgRNA targets (dark and light gray) flanking a TN3 Res1 core recognition sequence (core, orange). The two sgRNAs have a protospacer adjacent motif (PAM, red) distal orientation. mTN3-dCas9 fusions bind in positions around the core sequence allowing for mTN3 catalytic domain dimerization. The components identified by different hatching patterns in FIGS. 1B and 1C correspond with the identified hatching pattern in FIG. 1A.

FIGS. 2A-2F depict, in accordance with certain embodiments, the validation of iCas9 function and target site design using a yeast-based GFP-deletion assay. FIG. 2A depicts a diagram of chromosomally integrated dual-fluorescent reporter for detection of iCas9 function. The reporter contains GFP and mCherry coding regions transcribed from separate TEF1 promoters (arrows). iCas9 recognition sites flank the GFP expression cassette, wherein each site contains a left and right protospacers flanking a TN3 Res1 core sequence. Functional targeting of iCas9 results in GFP deletion generating GFP−, mCherry+ cells. FIG. 2B depicts a representative flow cytometry scatter plot for yeast expressing the reporter, iCas9, sgRNAs G and H after 96 hours of galactose induction of iCas9 expression. NFC is non-fluorescent channel. FIG. 2C depicts systematic analysis of sgRNA spacing on iCas9 function, as measured by GFP-deletion on flow cytometry. Inset shows spacing as measured from 5′ ends of sgRNAs flanking the core sequence. sgRNAs A-M are systematically spaced around the core sequence and distances ranging from 16-40 bp. sg(−) is a control guide not matching the target site, where the dashed line indicates background false-GFP-deletion. “Symmetric”, indicates left and right guides are positioned equal distances around the core site. “Asymmetric” guide combinations are at varying distances from the core. FIG. 2D depicts fluorescent microscopy of yeast expressing iCas9 and non-target guide, sg(−), or the 22 bp targeting pair, sg(G:H). GFP and mCherry dual-positive cells are orange on merge, while GFP-deletions appear as red only (GFP−, mCherry+). Scale bar is 20 μm. FIG. 2E depicts gel-electrophoresis of amplicons using primers flanking the reporter locus. The starting reporter results in a 5 Kilobase (Kb) PCR product and GFP-deletion results in a 4 Kb amplicon. Co-expression of iCas9 and sg(G:H) results in detectable DNA-deletion via formation of the 4 Kb product. FIG. 2F depicts sequencing of iCas9 target sites from isolated and sub-cloned deletion amplicons. Sequencing results (SEQ-1 to SEQ-5) aligned to the expected recombination product (EXPECT). Deletion products match the expected recombination sequence and are free of insertion deletion (indel) mutations. The components identified by different hatching patterns in FIGS. 2C and 2E correspond with the identified hatching pattern in FIG. 2A.

FIGS. 3A-3B depict, in accordance with certain embodiments, the detection of iCas9 function using an episomal deletion assay in human cells. FIG. 3A depicts dual-fluorescence plasmid systems contains an EF1α-HTLV promoter (arrow), iCas9 recognition sites (rectangles) flanking mCherry and a downstream GFP reading frame. iCas9 targeting results in deletion of mCherry and generation of a GFP only vector. FIG. 3B depicts GFP expression in HEK293T co-transfected with the GFP-mCherry reporter plasmid, iCas9 and guides targeting the recognition sites. Co-transfection of iCas9 and a non-target guide (−) resulted in no shift of GFP expression. However, targeting with 22, 30 and 40 bp sgRNA spacing's spacing shifted GFP by 2.8±0.7, 2.7±0.0.4 and 1.3±0.4% respectively. NS is Non-significant, * is P<0.05.

FIGS. 4A-4D depict, in accordance with certain embodiments, iCas9-targeted plasmid-to-plasmid recombination in human cells. FIG. 4A depicts a dual-plasmid reporter for detection of intermolecular recombination. A promoterless GFP-donor vector contains an iCas9 recognition site. A separate mCherry acceptor vector contains an EF1α-HTLV promoter with iCas9 target site and mCherry downstream. Recombination results in placement of GFP downstream of the promoter and mCherry-GFP dual-positive cells. FIG. 4B shows fluorescence of HEK293Ts co-transfected with dual-reporter plasmids, iCas9 and sgRNAs. Scale bar is 200 μm. FIG. 4C depicts flow cytometry scatter plots of plasmid-to-plasmid recombination experiments. Untransfected HEK293Ts (gray, lower left, LL) were used to define gates for GFP+ and mCherry+ (dashed lines). HEK293Ts were co-transfected with reporter vectors, iCas9 and non-targeting sg(−) (red) or sg(G:H) (blue). Targeting resulted in GFP-mCherry dual-positive cells (upper right, UR). FIG. 4D depicts fold-increase of GFP-mCherry dual-positive cells for iCas9 transfections. Targeting of GFP-donor and mCherry-acceptor with sg(G:H) results in a 10.6±0.5 fold-increase of dual-positive cells, results of recombination, at the target site compared to a control sgRNA sg(−).

FIGS. 5A-5F depict, in accordance with certain embodiments, multiplex-targeting of iCas9 enables genome integration in human cells. FIG. 5A depicts a genome integrated mCherry acceptor cassette contains an EF1α-HTLV promoter and downstream iCas9 recognition site with an mCherry coding sequence. Integration of GFP into the genomic acceptor cassette results in GFP+ cells. FIG. 5B depicts a design scheme for accessory targeting adjacent to the iCas9 core target site. Recombination between GFP-donor (green) and mCherry-acceptor (red) is coordinated by multiplex targeting of iCas9 binding. Accessory guide sites were targeted downstream of the iCas9 core site. Targeting of the + or − strand and varying distances (X bp) were tested. FIG. 5C depicts the fold-increase of GFP+ over sg(−) control. Targeting with iCas9 at the core site and downstream accessory 21 bp away resulted in 9.4±2.5 fold-increase of GFP+ cells. FIG. 5D depicts PCR detection of integration from isolated genomic DNA using primers flanking the recombination junction (inset by photo). “Mock” is a mock transfection of the mCherry-acceptor HEK293T cell line. iCas9 and GFP-donor were co-transfected with various guide combinations, (−) is a non-target guide, (G:H) is the 22 bp spacing without accessory guide, and (G:H:M) is 22 bp spacing with accessory targeting. FIG. 5E depicts alignments of sub-cloned and sequenced PCR products against the expected recombination product (EXPECT). SEQ-1 to SEQ-5 are free of indel mutations. FIG. 5F depicts alignments of sub-cloned and sequenced PCR products for Cas9WT-targeted NHEJ-mediated integration products. Some products contain indel mutations.

FIGS. 6A-6D depict, in accordance with certain embodiments, S. cerevisiae Reporter iCas9 and sgRNA vectors. FIG. 6A depicts a yeast genome integration vector with reporter for iCas9 function. The plasmid contains a HIS3 (histidine) prototrophic marker. URA3 homology arms (HAs) contain distinct StuI and ApaI sites. Digestion generates a linear plasmid capable of genome integration at the URA3 locus. The plasmid contains a constitutive mCherry cassette with a translation elongation factor 1 (TEF1) promoter. A constitutive enhanced GFP (eGFP) cassette is flanked by iCas9-sites (see FIG. 7). iCas9-sites are cloned into EcoRI and MluI restriction sites upstream and downstream of the eGFP cassette. The plasmid contains a ColE1 origin of replication and ampicillin selection marker for bacterial propagation. FIG. 6B shows p415-Gal1-iCas9, which is the episomal expression vector for iCas9. iCas9 is composed of mTN3 catalytic domain, glycine serine (GGS)6 linker and dCas9 (i.e. Cas9 D10A, H840A). A galactose inducible (GAL1) promoter controls expression of iCas9. The plasmid contains a Cen6-ARS yeast episomal replication origin and LEU2 (leucine) prototrophic marker for positive selection. FIG. 6C shows pYSG0-1C3, which is a cloning chassis for generating individual sgRNA cassettes. Guide oligonucleotide duplexes are cloned into SapI digested vector (highlighted on inset), wherein a small nucleolar-RNA 52 (SNR52, green) promoter is upstream and the S. pyogenes sgRNA hairpin structure is downstream (blue). The vector contains a ColE1 origin of replication and chloramphenicol resistance cassette. FIG. 6D shows pRS424-sgRNA(s), which is used for expression of guides in yeast. The yeast episomal vector contains a 2p origin of replication and TRP1 (tryptophan) prototrophic marker. SNR52 promoters drive expression of each sgRNA (e.g. sg(G:H) shown). Individual or multiplex guides are cloned into distinct EcoRI and SpeI sites.

FIG. 7 depicts, in accordance with certain embodiments, an iCas9-Site Design. Target sequence for iCas9 consists of a core TN3 Res1 sequence combined with randomized sequence with multiple protospacer adjacent motifs (PAMs) flanking. These enabled systematic spacing of sgRNA pairs. Icons indicate positioning of left (filled) and right (not shaded) sgRNA targets. (for specific iCas9-site and sgRNA sequences see supplemental sequences).

FIG. 8 depicts, in accordance with certain embodiments, an explanatory graphic for functional sgRNA spacings. A conceptual illustration of the effect of sgRNA spacings. The DNA helix is approximately 10.5 bp per helix turn1. Likewise, γΔ resolvase (a close homolog to TN3 resolvase) DNA-binding domains bind to the same helical face and present catalytic domains in a specific orientation with respect to the substrate DNA. This corresponds to functional sgRNA spacing of 22 bp (sg(G:H)) and 40 bp (sg(K:L). The 22 bp spacing positions 5′ end's of guides on the same helical face. However 30 bp (sg(I:J)) places left and right sgRNAs on the same face, but the opposite with respect to 22 bp. 40 bp results in placement of 5′ end of sgRNAs on the same face as 22 bp. Similar targeting patterns have been reported with FokI-dCas9 fusions, where the functional requirements of the FOKI restriction enzyme domains constrain functional sgRNA pairs to specific nucleotide spacings.

FIGS. 9A-9B depict, in accordance with certain embodiments, the effect of interdomain linkers on iCas9 function. FIG. 9A depicts the iCas9 primary structure, with N-terminus (N) and C-terminus (C). Both termini have SV40 nuclear localization sequences (NLS). A TN3 resolvase catalytic domain (mTN3) is upstream of a dCas9 coding region. A linker region is between mTN3 and dCas9. A series of amino acid sequences on iCas9 function was tested. These range from short glycine serine (Linker-1) to longer glycine serine (linker-2), previously described linkers for dCas9 fusions (XTEN3, Linker-3) and a novel fusion of glycine serine and XTEN (Linker-4). FIG. 9B depicts a yeast genome GFP-deletion assay with aforementioned linkers and functional sgRNA pairs sg(G:H), 22 bp; sg(K:L), 40 bp. sg(−) is a non-target control guide.

FIGS. 10A-10F depict, in accordance with certain embodiments, human cell reporter iCas9 and sgRNA Vectors. FIG. 10A depicts a ‘Traffic-light’ (TL) reporter for iCas9 function in human cells. A EF1α-HTLV promoter drives expression of mCherry and eGFP reading frames. mCherry is flanked by iCas9-sites. Deletion of mCherry results in cells with relative GFP+. A rabbit β-globin terminator is downstream of eGFP and mCherry. Sequences are cloned into a pUC19 backbone. FIG. 10B depicts pUC19-mCherry-acceptor (MA), which has an EF1α-HTLV promoter that drives expression of a mCherry fused with a puromycin resistance cassette. A single iCas9-site enables integration downstream of the promoter. FIG. 10C depicts a promoterless eGFP cassette with iCas9-site on the pSB1C3 backbone. eGFP is conditionally expressed when integrated at iCas9-sites. FIG. 10D depicts pKSBRV-1, which is a 2nd generation retroviral vector with mCherry-T2A-PuroR. A single iCas9-site is between mCherry and the Ef1α-HTLV promoter. After viral transduction, this functioned as the genomic reporter locus. FIG. 10E depicts a dual-targeted sgRNA expression vector. Human U6 promoters drive expression of each guide (e.g. sg(G:H), blue). FIG. 10F depicts a transient iCas9 expression vector. A CBH promoter drives expression of mTN3-(GGS)6-dCas9 (i.e. iCas9).

FIG. 11: Design of Accessory sgRNAs. Accessory sgRNAs as targeting the genomic reporter locus (blue) were targeted to the + or − strand at varying bp distances from sg(H) (X bp). Distances are listed by each guide. Targeted strand is that which is complementary to the guide sequence.

### DETAILED DESCRIPTION

Detailed aspects and applications of the disclosure are described below in the following drawings and detailed description of the technology. Unless specifically noted, it is intended that the words and phrases in the specification and the claims be given their plain, ordinary, and accustomed meaning to those of ordinary skill in the applicable arts.

In the following description, and for the purposes of explanation, numerous specific details are set forth in order to provide a thorough understanding of the various aspects of the disclosure. It will be understood, however, by those skilled in the relevant arts, that embodiments of the technology disclosed herein may be practiced without these specific details. It should be noted that there are many different and alternative configurations, devices and technologies to which the disclosed technologies may be applied. The full scope of the technology disclosed herein is not limited to the examples that are described below.

The singular forms “a,” “an,” and “the” include plural referents unless the context clearly dictates otherwise. Thus, for example, reference to “a step” includes reference to one or more of such steps.

As referenced herein, the spacing between sequences elements are measured as the bp distance between adjacent ends. For example, the spacing between accessories sgRNAs and the iCas9-site is the bp distance between the right guide of the iCas9-site (i.e. sg(H)) and the start of the accessory guide (e.g. sg(M) or (N)).

While clustered regularly interspaced short palindromic repeats (CRISPR) and CRISPR-associated (Cas) systems have made headlines as powerful tool for genome editing, site-specific recombinases are also powerful tools for genome engineering and synthetic biology. Site-specific recombinases are capable of facilitating DNA rearrangements with high predictability and specificity without incurring DSBs. These proteins possess the enzymatic machinery to facilitate transient DNA cleavage, strand-exchange and re-ligation without the need for high energy cofactors, DNA replication or DSB repair. Certain site-specific recombinases, such as ΦC31, are limited to specific ~30 bp recognition sites and are often used for integration at specific ‘landing pad’ or pseudo-site loci. To circumvent this, directed evolution has been employed to retarget recombinase substrate specificity. For instance, Karpinski et al. reported directed evolution of Cre recombinase to target conserved sequences Human Immuno-deficiency Virus (HIV) long-terminal repeats (LTRs). This system led to efficient and highly specific excision of the HIV provirus; however, nearly 150 rounds of directed evolution were required. Alternatively, recombinases have been retargeted by fusing catalytic-domains to zinc finger or transcriptional activator-like (TAL) DNA-binding domains. These techniques however require complex addition of heterologous DNA-binding domains.

The disclosure relates to a new tool for genome editing that takes advantage of the programmability of the CRISPR-Cas system for targeted gene editing while using the functionality of a site-directed recombinase. The disclosure reports that a fusion protein comprising a catalytically inactive Cas9 fused with the catalytic domain of a recombinase overcomes the limitations of both the CRISPR-Cas system and site-directed recombinases. The recombinase is a TN3 resolvase. The examples demonstrate the function of iCas9 using the native TN3 core sequence. Likewise, zinc finger recombinase literature has focused largely on targeting canonical core sequences. There have been conflicting reports about the versatility of this family of serine recombinases. Some reports indicate Gin recombinase, a TN3 resolvase homolog, is highly versatile. However, other reports indicate directed evolution and rationally targeted mutagenesis are required to retarget substrate specificity. The versatility of iCas9's core sequence could be increased by fusion with highly versatile PAM-variant Cas9s, such as xCas9 or Cas9 orthologs in certain embodiment.

In some aspects, the fusion protein comprises a catalytically inactive Cas9 and a catalytic domain of a hyperactive Tn3 transposon resolvase. For example, the fusion protein comprises a catalytically inactive Cas9 and a catalytic domain of a hyperactive Tn3 transposon resolvase, where a first linker connects the C-terminus of the catalytic domain of the recombinase to the N-terminus of the catalytically inactive Cas9. The fusion protein also comprises a first nuclear localization signal, where a second linker connects the first nuclear localization signal to the C-terminus of the catalytically inactive Cas9 or the N-terminus of the catalytic domain of the recombinase. In some embodiments, the fusion protein further comprises a second nuclear localization signal wherein the first nuclear localization signal adjacent to the C-terminus of the catalytically inactive Cas9 and the second nuclear localization signal is adjacent to the N-terminus of the catalytic domain of the recombinase. Such embodiments of the fusion protein further comprise a third linker, wherein the second linker connects the first nuclear localization signal to the C-terminus of the catalytically inactive Cas9 and the third linker connects the second nuclear localization signals to the N-terminus of the catalytic domain of the recombinase. In some aspects, the linkers are flexible glycine serine linkers. For example, the amino acid sequence of the linker comprises repeats of GGS, SGSETPGTSESATPES (SEQ ID NO. 120), GGSGGSGSETPGTSESATPES (SEQ ID NO. 121), or combinations thereof. In certain embodiments, the nuclear localization signal is from SV40.

In a particular embodiments, the fusion protein is a hyperactive mutant TN3 resolvase fused to dCas9 with an amino acid sequence set forth in SEQ ID NO. 1, or having at least 90%, at least 92%, at least 94%, at least 95%, at least 96%, at least 97%, at least 98%, or at least 99% sequence similarity thereto, or the nucleic acid sequence set forth in SEQ ID NO. 2 having at least 80%, at least 85%, at least 90%, at least 92%, at least 94%, at least 95%, at least 96%, at least 97%, at least 98%, or at least 99% sequence similarity (also referred to herein as “iCas”). The disclosure also encompasses the method of producing iCas9.

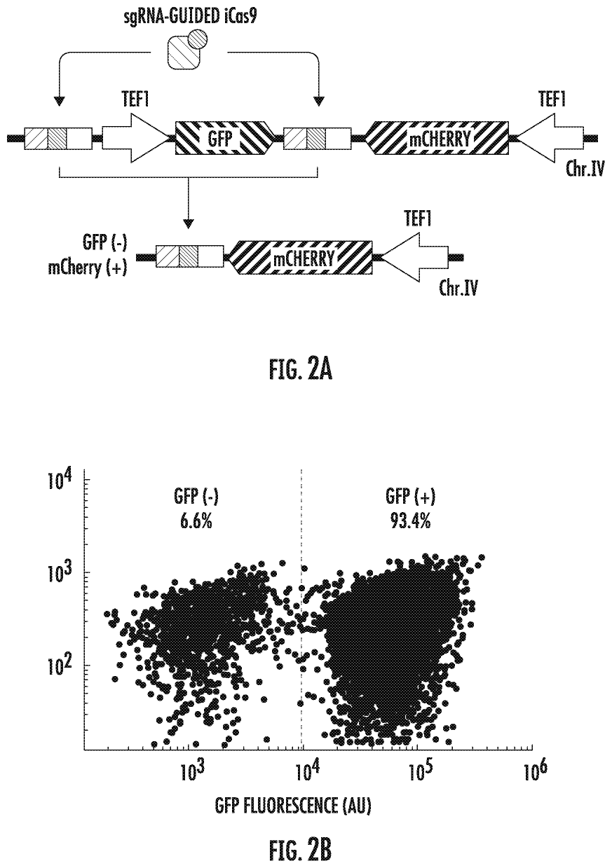

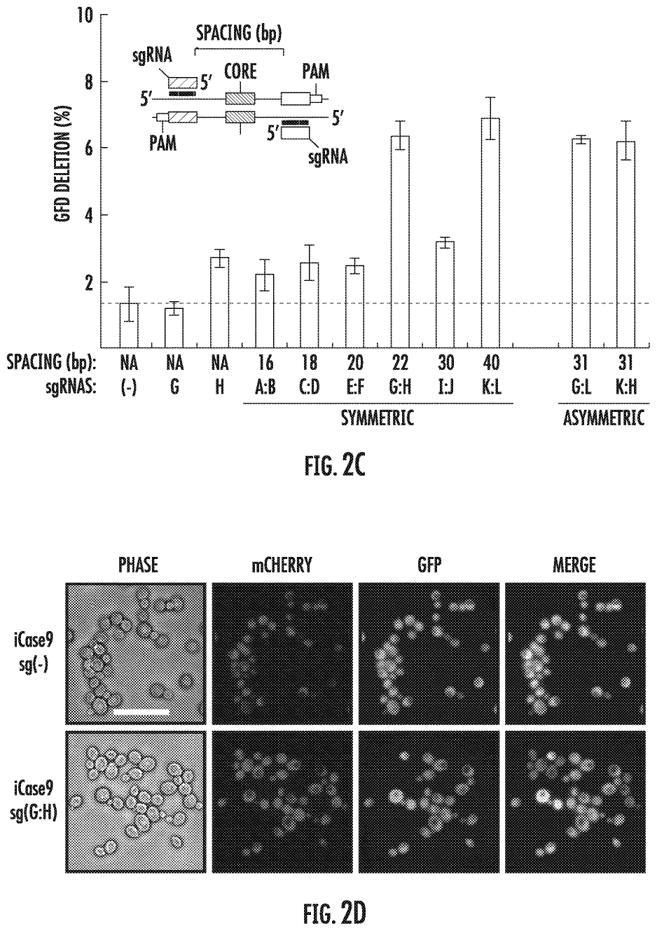

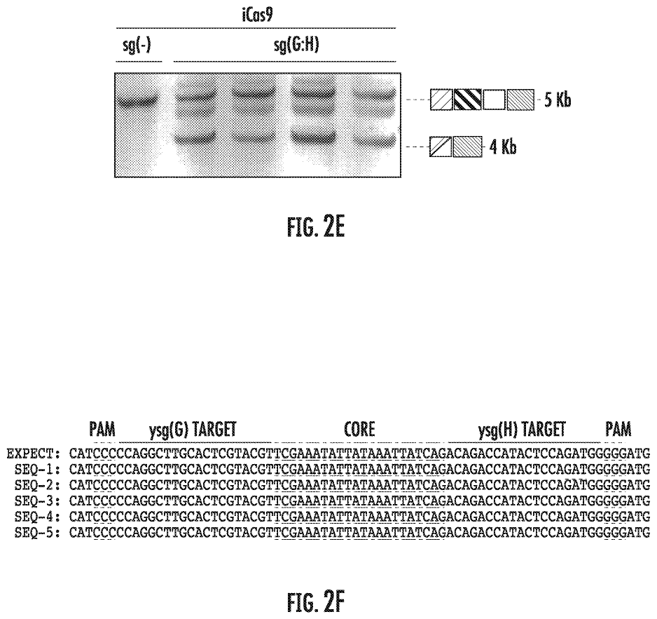

As shown in the examples, iCas9 is capable of targeted DNA deletion and targeted DNA insertion of the genome of multiple eukaryotic hosts, ranging from yeast to human cells. However, unlike other recombinant Cas9, the optimal spacing between the guide sequences is greater than 20 bp, as shorter spacing resulted in little to no recombination (FIG. 2).

The yeast experiments (see Example 3) identified optimal symmetric spacing's of 22 and 40 bp and asymmetric spacing's of 31 bp. Interestingly, this is consistent with the Watson-Crick DNA structure being 10.5 bp per helix turn combined with the requirement for co-localization of mTN3 catalytic domains to the same helical face of the DNA molecule (See FIG. 8). Furthermore, optimal sgRNA spacing of 22 bp is corroborated by zinc finger mTN3 fusions, which have an optimal spacing of 20-22 bp. In general, this is supported by FokI-dCas9 fusions that use 15 or 25 bp spacings, where these spacings match the requirement for FokI dimerization on opposite DNA helical faces.

As shown in Example 4, iCas9 is capable of targeted DNA deletion and targeted DNA insertion in human cells, and the results confirmed the functionality of the 22 bp sgRNA spacing. The experiments in human cells also found 30 bp to be functional, which is consistent with previous reports using analogous recombinase-Cas9 designs. These altered spacing stringencies may be due to the use of supercoiled plasmids as substrates, which may have different spacing requirements than linear genomic DNA.

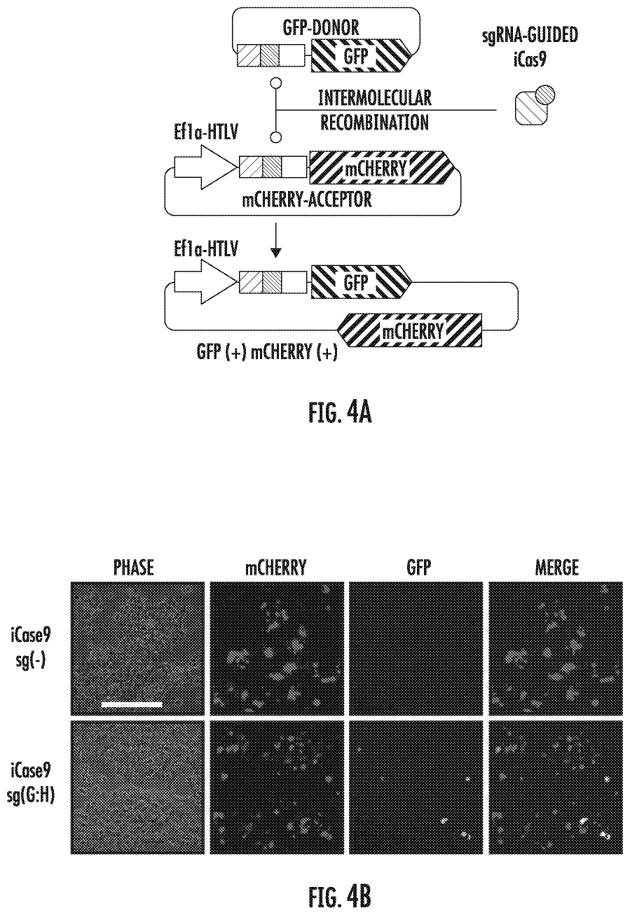

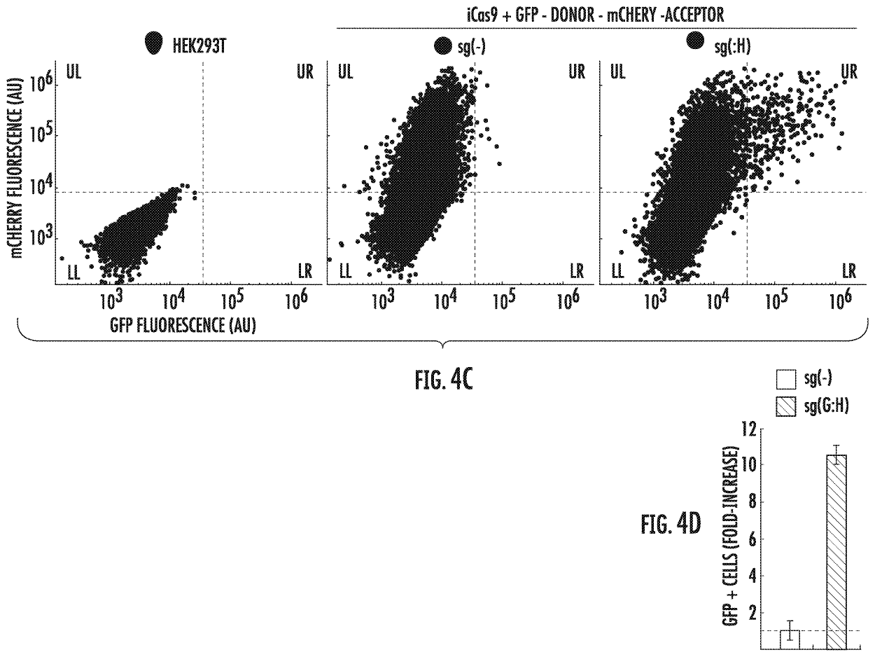

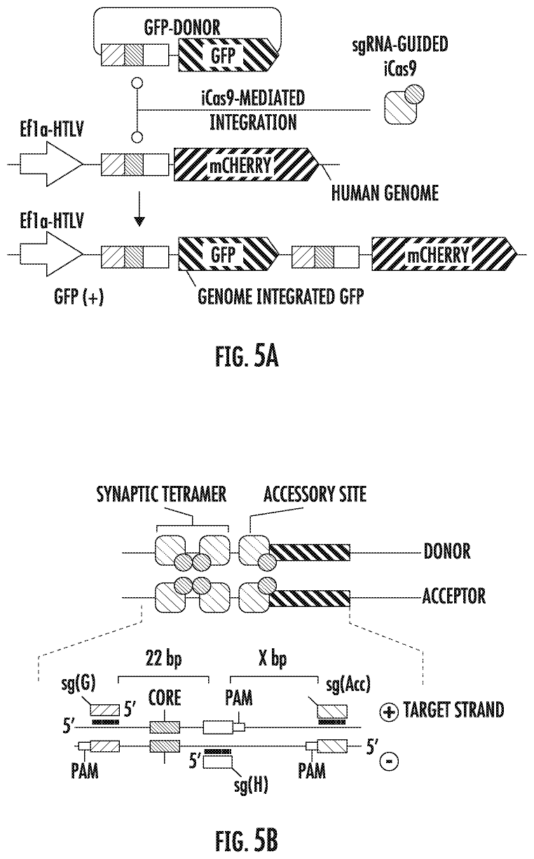

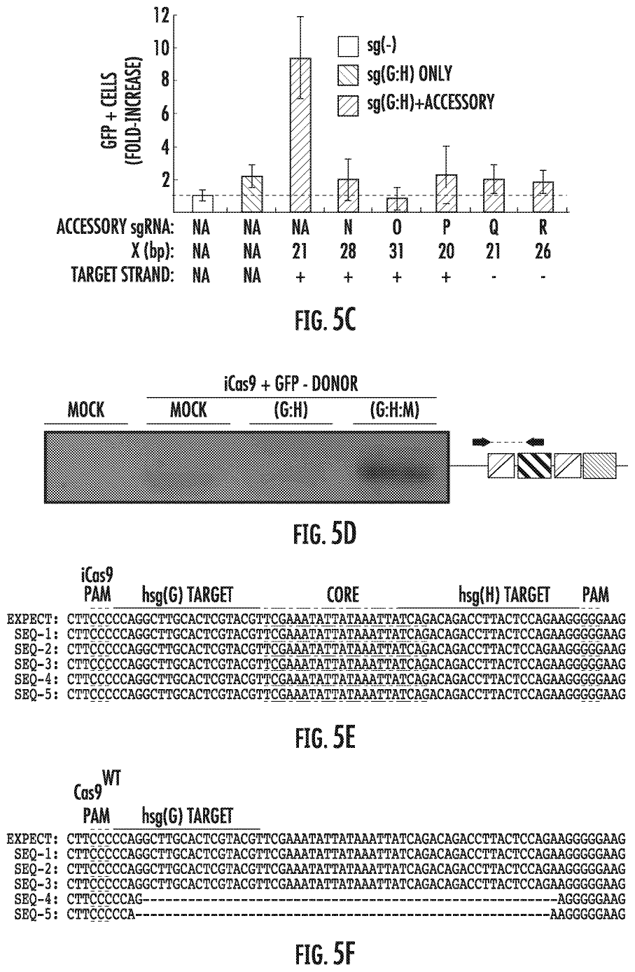

Accordingly, iCas9 may be a useful tool for targeted DNA integration. While previous reports have fused dCas9 to recombinase domains, these systems were incapable of genomic integration. For the first time, iCas9's ability to target intermolecular recombination has been validated, and it was through the use of an episomal assay described herein. The experimental design separated the assay from constraints of targeting the human genome, such as being long linear DNAs constrained in 3D space and compacted into different nuclear regions. Although the assay confirmed iCas9 is capable of targeting linear eukaryotic genomic DNA (FIG. 2) and can direct plasmid-to-plasmid recombination (FIG. 4), Donor-DNA-iCas9 complexes still did not interact with the genomic target locus. To address this, the guide sequence vector design was adopted to a scheme of accessory target site binding, wherein sgRNAs are targeted adjacent to the core sequence guides. Accessory binding sites for TN3 resolvase have been implicated in regulating 3D presentation of recombinase subunits, local DNA supercoiling and result in improved recombination efficiency. A tiling of sgRNAs was designed to test if accessory binding sites can be recapitulated with iCas9. Interestingly, the verified functionality of 21 bp spacing and sgRNA orientation of accessory sg(M) approximates the 22 bp spacing observed between the Res1-core and adjacent accessory binding sites native to TN3 transposon (FIG. 5).

iCas9 targeting of endogenous loci can be accomplished through a mixture of multiplex sgRNA design and development of novel-iCas9 derivatives targeting new core sequences, for example “pseudo-core” sites. Because each sgRNA guides an individual iCas9 to the target locus, multiplex targeting is necessary to achieve dimerization and tetramerization. For example, two sgRNA guides would guide dimerization, while four sgRNA guides would guide tetramerization. Targeting with more pairs of sgRNAs, for example, with 6 sgRNA guides would result in hexamerization.

Also described herein are dimer and tetramer of the recombinant Cas9. The dimer of the recombinant Cas9 refers to the fusion protein in a dimerized state, where the dimer is bound to a DNA molecule and a single guide RNA (sgRNA) bound to the catalytically inactive Cas9 portion of the fusion protein. Accordingly, the dimer of the fusion protein comprises two fusion proteins, two sgRNAs, and the DNA molecule. The DNA molecule is a target DNA that comprises binding sites for two single guide RNAs (sgRNA), where the distance between the binding sites for the two sgRNAs is at least 21 bp or at least 22 bp apart, for example, 22 apart, 30 bp apart, 31 bp apart, 40 bp apart, or 44 bp apart. In some aspects, the fusion protein (monomeric units of the dimer) is bound to the same strand of the DNA molecule; in other aspects, they are bound to an opposite strand of the DNA molecule. The tetramer of the recombinant Cas9 refers to the fusion protein in a state where a first dimer of the fusion protein is bound to a second dimer of the fusion protein. Accordingly, the tetramer of the fusion protein comprises four fusion proteins, four sgRNAs, and the DNA molecule. The first dimer and the second dimer are bound to same strand of the DNA molecule in same aspects or are bound to an opposite strand of the DNA molecule in other aspects.

Since iCas9 does have its own fused recombinase functionality, iCas9 may be used for therapeutic purposes or generation of new cell lines, where double-stranded DNA lesions caused by wild type Cas9 can lead to large, multiple kilobase, deletions, insertions, and complex rearrangements. Since iCas9 does not directly rely on DSBs repair pathways such as NHEJ and HR, it reduces the likelihood of precipitating unwanted mutations. Furthermore, mTN3 catalytic domains of iCas9 require paired targeting by sgRNAs (FIG. 2C), it follows that iCas9 should have higher specificity than canonical CRISPR-Cas9 editing techniques that rely on single or double stranded DNA breaks. Moreover, canonical CRISPR-Cas9 editing strategies rely on endogenous DNA repair. This may be detrimental to editing some cell lines recalcitrant to DNA repair. Previous reports have demonstrated the role cell cycle plays in homologous recombination. This has largely limited CRISPR-targeted editing techniques in post-mitotic cells. This may prevent ex vivo editing of patient primary cells. Likewise, it has been shown in embryonic stem cells and epithelial cells that P53 may inhibit repair and survival in cells with CRISPR-targeted DNA lesions. DSB-dependent editing results in an upregulation of P53 and apoptosis of edited populations. While suppression of P53 results in increased editing efficiencies, transient inhibition of P53 may increase tumorigenic potential of the edited cell population. This is an important consideration when developing edited cell populations for cell therapy applications. Since iCas9 utilizes mTN3 catalytic domains for recombination, it avoids the requirement for endogenous DNA repair and may be helpful in editing cell types recalcitrant to DNA manipulations.

iCas9 may also be used in the field of synthetic biology for the construction and implementation of recombinase-based gene networks. Recombinase based gene networks are of increasing interest to synthetic biology. These systems can integrate multiple biological inputs and turn them into saved ‘DNA memory’. Recombinase based logic can be constructed in a way to imbue biological systems with Boolean logic functions or even 8-bit memory. These systems are capable of robust function but require coexpression of multiple recombinases and placement of sites corresponding to each recombinase to generate single circuits. iCas9 could enable the generation of RNA-programmed recombinase-based gene networks, wherein different sgRNAs could target different recombinase operations. Unlike previous iterations of recombinase-based gene circuitry, iCas9 systems would only require coexpression of multiple sgRNAs instead of separate recombinases. Numerous sgRNAs could be easily programmed and placed under control of inducible promoters to create circuits that predictably and combinatorically restructure in response to environmental or physiological cues.

In another aspect, the disclosure is directed to methods of using a Cas9 fusion protein (for example, iCas9) for targeted DNA deletion or targeted DNA insertion in a eukaryotic genome. Also disclosed are assay kits and methods for evaluating the ability of a Cas9 fusion protein for targeted DNA deletion and/or targeted DNA integration in eukaryotic cells. In certain embodiments, the assay kits and methods are for evaluating the ability of a Cas9 fusion protein for targeted DNA deletion and/or targeted DNA integration in eukaryotic cells, for example human cells, that is independent of the constraints of targeting the human genome.

In some aspects, the kit for evaluating a recombinant Cas9's ability for targeted DNA deletion in an eukaryotic genome comprises a first expression vector comprising an expression cassette for expressing the recombinant Cas9, a second expression vector encoding guide sequences, and a third expression vector that identifies a target sequence for deletion.

In some embodiments, the kit for evaluating a recombinant Cas9's ability for targeted DNA insertion in an eukaryotic genome comprises a first expression vector comprising an expression cassette for expressing the recombinant Cas9, a second expression vector encoding guide sequences, a third expression vector encoding a acceptor sequence, wherein the third expression vector is a vector that integrates the acceptor sequence into the eukaryotic genome (for example, a retroviral vector), and a fourth expression vector encoding the donor sequence. The first expression vector, the second expression vector, the third expression vector, and the fourth expression vector enable expression in an eukaryotic organism.

In one embodiment, the recombinant Cas9 expressed by the first expression vector is a catalytically inactive Cas9 fused to a catalytic domain of a recombinase. The second expression vector comprises a first single guide RNA (sgRNA) sequence and a second sgRNA sequence. The third expression vector comprises an oligonucleotide encoding a Cas9 site. The third expression vector in the kit for evaluating the ability for targeted DNA deletion comprises the target sequence for deletion and at least one oligonucleotide encoding a Cas9 site, wherein the target sequence for deletion is flanked by the at least one oligonucleotide encoding the Cas9 site. The third expression vector in the kit for evaluating the ability for targeted DNA insertion further comprises an acceptor sequence, wherein the acceptor sequence is upstream of the oligonucleotide encoding the Cas9 site, and a promoter sequence, wherein the promotor sequence drives expression of the acceptor sequence. For the kit for evaluating the ability for targeted DNA insertion, the fourth expression vector is promotorless and comprises a donor sequence and an oligonucleotide encoding the Cas9 site, wherein the donor sequence is downstream of the Cas9 site.

The Cas9 site comprises a core sequence that is recognized by the catalytic domain of the recombinase; a sequence complementary to the first sgRNA sequence that is upstream of and adjacent to the core sequence; a sequence complementary to the second sgRNA sequence that is downstream of and adjacent to the core sequence; and at least two protospacer adjacent motif sequences. Of the at least two protospacer adjacent motif sequences, at least one protospacer adjacent motif sequence is upstream of the sequence complementary to the first sgRNA sequence, and at least one protospacer adjacent motif sequence is downstream of the sequence complementary to the second sgRNA sequence. The distance between the sequence complementary to the first sgRNA sequence and the sequence complementary to the second sgRNA is at least 22 bp apart.

In some embodiments, the second expression vector comprises a third sgRNA sequence and the Cas9 site further comprises an accessory site sequence. The accessory sequence comprises a sequence complementary to the third sgRNA and a protospacer adjacent region distal to the third sgRNA. The distance between the accessory sequence and the sequence complementary to the second sgRNA sequence is at least 21 bp. In other embodiments, the Cas9 site further comprises an accessory site sequence. Thus, the kit further comprises a fifth expression vector that comprises a third sgRNA sequence. The accessory sequence comprises a sequence complementary to the third sgRNA and a protospacer adjacent region distal to the third sgRNA. The distance between the accessory sequence and the sequence complementary to the second sgRNA sequence is at least 21 bp.

In some implementations, the distance between the accessory sequence and the sequence complementary to the second sgRNA sequence is 21 bp.

In some implementations, the sequence complementary to the first sgRNA sequence and the sequence complementary to the second sgRNA sequence on the third expression vector is 22 bp apart. In one aspect, the sequence complementary to the first sgRNA sequence and the sequence complementary to the second sgRNA sequence on the third expression vector is 30 bp apart and the eukaryotic genome is a human genome. In another aspects, the sequence complementary to the first sgRNA sequence and the sequence complementary to the second sgRNA sequence on the third expression vector is 31 bp apart and the eukaryotic genome is a yeast genome. In certain implementations, the sequence complementary to the first sgRNA sequence and the sequence complementary to the second sgRNA sequence on the third expression vector is 40 bp apart.

In certain implementations where the eukaryotic genome is a yeast genome, the oligonucleotide encoding the Cas9 site comprises a nucleic acid sequence set forth in paragraph

In certain implementations where the eukaryotic genome is a human genome, the oligonucleotide encoding the Cas9 site comprises a nucleic acid sequence set forth in SEQ ID NO. 116, SEQ ID NO. 117, SEQ ID NO. 118 or SEQ ID NO. 119.

The disclosure is also directed to methods of deleting a target sequence from the genome in an eukaryotic cell. The methods comprise introducing into the cell a first nucleotide sequence encoding a recombinant Cas9; introducing a first oligonucleotide sequence encoding a first single guide RNA (sgRNA) sequence and a second oligonucleotide sequence encoding a second sgRNA sequence; coexpressing the nucleotide sequence, the first oligonucleotide sequence, and the second oligonucleotide sequence in the eukaryotic cell to generate a transformed eukaryotic cell; and culturing the transformed eukaryotic cell to remove the region of target sequence from the genome of the cultured eukaryotic cell.

The disclosure additionally is directed to methods of inserting an extraneous sequence into a target region of a genome in a cell. The method comprises introducing into the cell a first nucleotide sequence that encodes the recombinant Cas9 protein described; introducing a first oligonucleotide sequence encoding a first sgRNA sequence, a second oligonucleotide sequence encoding a second sgRNA sequence, and a third oligonucleotide encoding a third sgRNA sequence; introducing a second nucleotide sequence encoding the extraneous sequence and a recognition site sequence for a recombinant Cas9 protein described herein; coexpressing the first nucleotide sequence, the first oligonucleotide sequence, the second oligonucleotide sequence, the third oligonucleotide sequence, and the second nucleotide sequence in the eukaryotic cell to generate a transformed eukaryotic cell; and culturing the transformed eukaryotic cell to insert the extraneous sequence into the genome of the cultured eukaryotic cell at the site of the target region. The recognition site is proximal to the extraneous sequence, and the recognition sequence comprises a sequence complementary to the region of the genome comprising the target region and at least 21 bp from the 3′ end of the target region.

The first sgRNA sequence is complementary to the 5′ end of a target sequence. The second sgRNA is complementary to the 3′ end of the target sequence. The target sequence also has a protospacer adjacent motif that is adjacent to and proximal to its 5′ end and a protospacer adjacent motif that is adjacent and distal to its 3′ end. The distance between the 5′ end of the target sequence and the 3′ end of the target sequence is at least 22 bp. The region of the target sequence between the 5′ end of the target sequence and the 3′ end of the target sequence comprises a sequence recognized by the catalytic domain of the recombinase of the recombinant Cas9 protein described herein. For the methods of inserting an extraneous sequence into a target region of a genome in a cell, the third sgRNA sequence is complementary to a sequence in the genome of the cell that is at least 20 bp from the 3′ end of the target region. In some aspects, the third sgRNA sequence is complementary to a sequence in the genome of the cell that is 20 bp or 21 bp from the 3′ end of the target region. The sequence in the genome of the cell that is at least 20 bp from the 3′ end of the target region comprises a protospacer adjacent motif distal to the sgRNA sequence.

In one implementation of the methods, the distance between the 5′ end of the target sequence and the 3′ end of the target sequence is 22 bp. In another implementation, the distance between the 5′ end of the target sequence and the 3′ end of the target sequence is 30 bp. In still another implementation, the distance between the 5′ end of the target sequence and the 3′ end of the target sequence is 31 bp. In yet another implementation, the distance between the 5′ end of the target sequence and the 3′ end of the target sequence is 44 bp.

The methods described herein do not cause off target mutations, nucleotide insertions, and/or nucleotide deletions, which are problems encountered when attempting to alter the genome with wildtype Cas9. In some aspects, the portion of the genome is deleted independent of the cell's endogenous DNA repair mechanism. For example, the portion of the genome is deleted by triggering non-homologous end joining.

### ILLUSTRATIVE, NON-LIMITING EXAMPLE IN ACCORDANCE WITH CERTAIN EMBODIMENTS

The disclosure is further illustrated by the following examples that should not be construed as limiting. The contents of all references, patents, and published patent applications cited throughout this application are incorporated herein by reference in their entirety for all purposes.

### 1. Methods

a. Bacterial Culture:

Molecular cloning was conducted using E. coli NEB-10-Beta (New England Biolabs, NEB). LB Miller Medium (Sigma Aldrich, Sigma) was supplemented with appropriate antibiotics for plasmid maintenance: Ampicillin (100 μg/ml), or Chloramphenicol (30 μg/ml). E. coli were cultured at 37° C.

b. Yeast Culture:

All yeast was cultured at 30° C. S. cerevisiae YPH500 were propagated on YPD agar plates and in liquid medium containing glucose. Liquid cultures were shaken at 250-300 RPM. Yeast minimal dropout media contained either 2% glucose or 2% galactose with 1% raffinose and necessary amino acid dropout solutions (Clonetech). Yeast were made competent using the Zymo competent yeast kit and transformed using manufacturer protocol. Genomic integrations and plasmid transformations were selected for on yeast minimal dropout plates with amino acid combinations necessary for selection. Yeast were cultured in liquid yeast dropout media necessary for plasmid selection.

c. Mammalian Cell Culture:

HEK293T cells (ATCC CRL-3216) were cultured on poly-L-ornithine (PLO) (Sigma) coated plates and maintained in Dulbecco's modified eagle medium supplemented with 10% (v/v) fetal bovine serum (FBS) and 1% (v/v) penicillin-streptomycin (all from ThermoFisher). Cells were maintained in a 37° C. incubator with 5% CO₂ and passaged once ~80% confluent.

d. Molecular Cloning:

iCas9 (TN3-GGSx6-dCas9) was constructed by fusion of a previously described hyperactive mutant recombinase (TN3 G79S, D102Y, E124Q). The resolvase catalytic domain (AA1-148) was linked to Cas9 D10A, H840A with a flexible glycine serine (GGSx6) linker. N- and C-terminal SV40 nuclear localization sequences with small glycine serine linkers (GGSx1) were added to facilitate nuclear entry. The coding region for the hyperactive TN3 mutant resolvase was synthesized as a human codon optimized gBlock by Integrated DNA technologies (IDT). The gBlock was sub-cloned into a dCas9 derivative of p415 Gal1-Cas9 (Addgene #43804). The mTN3 catalytic domain along with D10A and H840A mutations to Cas9 were added using PCR primers containing SapI sites (Table 2). The amino acid sequence of iCas9 is set forth in SEQ ID NO. 1. The nucleic acid sequence of iCas9 is set forth in SEQ ID NO. 2.

Purified PCR products were digested with SapI and gel-extracted using the Sigma-Aldrich gel-extraction kit. iCas9 was assembled in XbaI-XhoI sites of p415 Gal1-Cas9. The resulting p415 Gal1-iCas9 vector also contains a Cen6 origin of replication and a leucine prototrophic marker. For expression in human cells iCas9 was PCRed with primers adding AgeI and MfeI upstream and downstream respectively. iCas9 was cloned into a modified pX330 with guide expression cassette removed. Digested and gel-extracted iCas9 PCR products were ligated with AgeI and EcoRI digested pX330. The resulting vector contains a CBH-promoter driving iCas9 expression.

sgRNA guides were synthesized as pairs of oligonucleotides. 5′ phosphates were added to oligonucleotides by incubating lug total of top/bottom oligonucleotides in 50 μl reactions containing 1×T4 DNA Ligase Buffer and 10 units of T4 Polynucleotide Kinase (T4 PNK) at 37° C. overnight (Tables 1 and 2). Oligonucleotides were duplexed by heating the kinase reactions to 90° C. on an aluminum heating block for 5 minutes followed by slowly returning the reaction to room temperature (25° C.) over approximately 1 hour. Following duplexing, guides were ligated into respective vectors.

Yeast sgRNA expression cassettes, were constructed by cloning oligonucleotide duplexes into, pSB1C3 containing an SNR52 promoter with inverted SapI sites and an sgRNA hairpin recognized by S. pyogenes Cas9. Pairs of sgRNAs were then amplified with primers adding EcoRI and SapI, or SapI and SpeI sites. Purified PCR product were then digested with respective restriction enzymes, heat inactivated and ligated into EcoRI and SpeI digested pRS424. The resulting vector contains pairs of yeast sgRNA cassettes with a 2p origin of replication and tryptophan prototrophic marker.

Humanized sgRNAs were cloned into a modified pSB1C3 vector containing a human U6 promoter, inverted BbsI sites and a S. pyogenes recognized sgRNA hairpin (Sequence derived from pX330). Pairs of sgRNAs were then amplified with primers adding EcoRI and SapI, or SapI and XbaI sites. Purified PCR product were then digested with respective restriction enzymes, heat inactivated and ligated into EcoRI and XbaI digested pUC19. The resulting vector contains pairs of human sgRNA expression cassettes.

The Yeast Genomic Integration Vector (pMG) was generated using vectors previously described. Tef1 promoters drive constitutive expression of GFP and mCherry. To integrate into the yeast genome, one to two micrograms of pMG was digested with ApaI in 50 μl reactions for one hour or more at 37° C. Five microliters of the restriction product was transformed into competent YPH500 using protocol from Zymo Competent Yeast Kit (Zymo). Integrant were selected for by plating on histidine dropout plates.

To clone iCas9-target sequences into pMG, sites were synthesized as overlapping oligonucleotides. 5′ phosphates were added to oligonucleotides by incubating lug of top/bottom oligonucleotides in 50 μl reactions containing 1×T4 DNA Ligase Buffer and 10 units of T4 Polynucleotide Kinase (T4 PNK) at 37° C. overnight. Oligonucleotides were duplexed by heating the kinase reactions to 90° C. on an aluminum heating block for 5 minutes followed by slowly returning the reaction to room temperature (25° C.) over approximately one hour. Following duplexing, sites were ligated into EcorI and MluI sites surrounding GFP.

e. Mammalian Cell Transfections

HEK293T cells were seeded at 1.8×105 cells/well in PLO coated 24-well plate and transfected 24 hours post-passage at ~80% confluency. For plasmid-plasmid assays, 300 ng of iCas9, 100 ng of GFP-encoding donor vector (FeGFP-1C3), 100 ng of mCherry-expressing target vector (pUC:EAMP), and 100 ng sgRNA expression vectors were transfected per well using 1.5 μl Lipofectamine 3000 and 1 μl P3000. For genome integration experiments, 300 ng iCas9 expression vector, 100 ng GFP-encoding donor vector (FeGFP-1C3), 100 ng pIRFP670 and 100 ng sgRNA cassette(s) were transfected using 1.5 μl Lipofectamine 3000 and 1 μl P3000. pIRFP670 was co-transfected as a control with samples at >50% transfection efficiency.

f. Retrovirus and Stable Cell Line Generation

HEK293T cells were passaged to four PLO coated 100 mm culture plates in Opti-MEM reduced serum medium plus GlutaMAX and supplemented with 1 mM sodium pyruvate and 10% (v/v) FBS (all from ThermoFisher). To generate recombinant retroviruses, HEK 293T cells were transfected with the pKSBRV-1 transgene and packaging plasmids (pUMVC and pVSVG). 9 μg pKSBRV-1, 6 μg pUMVC, and 3 μg pVSVG expression plasmids were transfected per plate using 28 μl Lipofectamine 3000 and 36 μl P3000 (ThermoFisher). Media was changed 6 hours post-transfection and lentivirus containing supernatant was collected at 24 hours and 54 hours. Conditioned media was filtered using 0.45 μm filter and lentiviral particles were concentrated using Lenti-X (Takara Bio). HEK293T cells were then infected with the viruses followed by puromycin selection 48 hours later at a concentration of 0.75 μg/mL. Following selection for 2 weeks, cells were FACS sorted for the upper 50% of mCherry expressing cells to generate a pure population of cells stably expressing the transgene.

g. In Yeast GFP-Deletion Assay

To assay iCas9 function, YPH500 Ura3(MGaa) with p415 Gal1-iCas9 and with various pRS424(guide pairs) were cultured in 3 ml YP -Leu, -Trp with 2% Glucose. After 24 hours, 5 μl of the stationary phase culture was used to inoculate 3 ml of YP -Leu, -Trp with 2% Galactose, 1% Raffinose. Cell were diluted down (5 μl saturated culture in 3 ml media) at 48-hour intervals. Cells were analyzed by flow cytometry and fluorescent microscopy after 96 hours of galactose induction. Genomic DNA was also prepared after galactose induction.

h. Flow Cytometry

All flow cytometry was conducted on an Accuri C6 Flow Cytometer (BD Biosciences, CA). Samples were gated by consistent forward scatter (FSC) and side scatter (SSC) and 10,000 events within the FSC/SSC gate were collected. A 488 nm laser excitation and a 530±15 nm emission filter was used for GFP fluorescence determination. Flow cytometry files were analyzed using manufacture software and in MatLab (The MathWorks). Flow cytometry of HEK293T cells was conducted 72 hours post-transfection. Briefly, cells were dissociated using Accutase (ThermoFisher), washed with PBS, and analyzed using a BD Accuri C6 cytometer (BD Biosciences). GFP-positive cells were measured compared to transfections with a non-target sgRNA.

i. Fluorescent Microscopy

200 μl of stationary phase cultures of yeast were spun down at 4000*g for 2 minutes and washed once in 1×PBS solution. Following washing, cells were concentrating by resuspending in 10-20 μl of 1×PBS. 1-2 μl of cell solution was placed on glass microscope slides and visualized on a Nikon Ti-Eclipse inverted microscope with and LED-based Lumencor SOLA SE Light Engine with appropriate filter sets. GFP was visualized with an excitation at 472 nm and emission at 520/35 nm using a Semrock band pass filter. mCherry was visualized with excitation at 562 nm and emission at 641/75 nm. Constant exposure times, LUT and image gain adjustments were applied to microscopy data. HEK293T cells were imaged directly on TC plates 72 hours after transfection.

j. Genomic DNA Isolation and PCR Analysis of GFP Deletions

Yeast genomic DNA was prepared using the Zymo yeast genomic DNA preparation kit using the manufacturer's protocol with phenol-chloroform steps included. To assay genomic deletion, PCR was conducted using Phusion DNA polymerase (New England Biolabs). Annealing temperatures and extension times were calculated using the manufacturer's protocol. PCR products were visualized via 0.8% agarose gel electrophoresis. Human cell genomic DNA was prepared 72 hours post-transfection using the Qiagen DNEASY kit using the manufacturer protocol. PCR was conducted on 250 ng of genomic DNA with primers target the integration junction. Products were resolved on a 2% agarose.

k. Sequencing of Deletion and Integration Products

Following gel resolution of amplicons, deletion bands were gel-extracted using the Gen Elute gel extraction kit (Sigma-Aldrich) using the manufacturer's protocol. Following extraction, products with phosphorylated via incubation in 50 μl reactions with T4 PNK and 1×T4 DNA ligase buffer. Reactions were heat inactivated and ligated in equimolar ratio to SmaI cleaved and dephosphorylated pUC19. Ligations were transformed into chemically competent NEB10B E. coli and plated on Ampicillin Plates supplemented with 40 μl X-Gal solution (Promega). White colonies were picked and prepared using GeneElute Plasmid Preparation kit (Sigma-Aldrich). 300 ng of plasmid DNA was sequenced via DNASU's Sanger Sequencing Core facility.

2. Design of iCas9 and Guide Sequences for RNA-Guided Targeting of iCas9

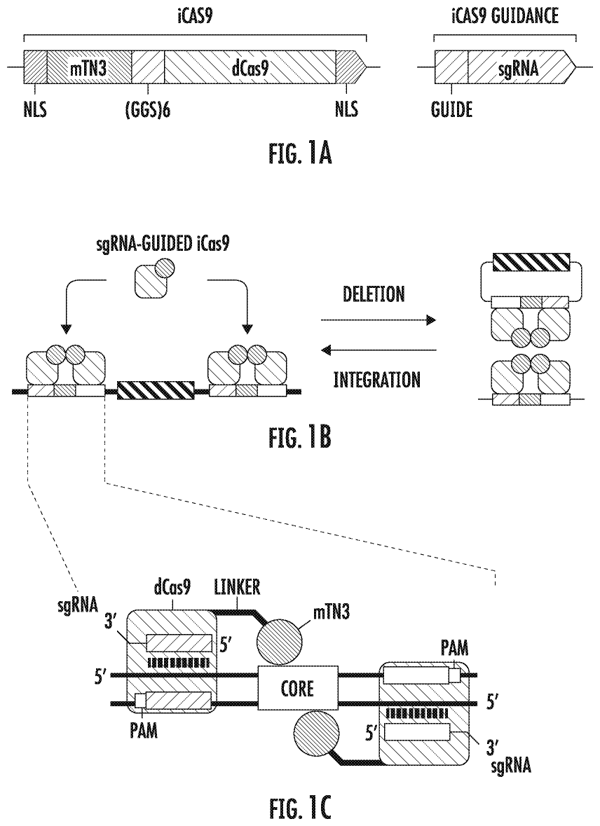

The design of iCas9 followed several general principles. First, the fusion of catalytically inactive Cas9 (dCas9) with a hyperactive mutant TN3 resolvase (mTN3) was accomplished by addition of the N-terminal resolvase catalytic domain to the N-terminus of dCas9 (FIG. 1A). These domains were separated by a flexible glycine serine (GGSx6) linker. To facilitate nuclear entry, SV40 nuclear localization sequences (NLS) were added on both the N- and C-termini. The choice of mTN3 was motivated by previous studies that showed mTN3 zinc finger fusions were capable of DNA deletion and integration (FIG. 1B). Finally, previous work demonstrated FokI-dCas9 fusion proteins dimerize when pairs of sgRNAs were targeted in a PAM-distal orientation. This suggested that mTN3's N-terminal heterologous fusion with dCas9 are presented adjacent to the 5′ end of the sgRNA bound to a protospacer DNA. Furthermore, solved protein structures for Streptococcus pyogenes Cas9 μlace the N-terminus closer to the 5′ end of the sgRNA than the C-terminus. Collectively, structural information and previous FokI-dCas9 results strongly suggest that a PAM-distal protospacer orientation flanking a mTN3 core recognition site should enable RNA-guided targeting (FIG. 1C).

### 3. Validation Using Yeast

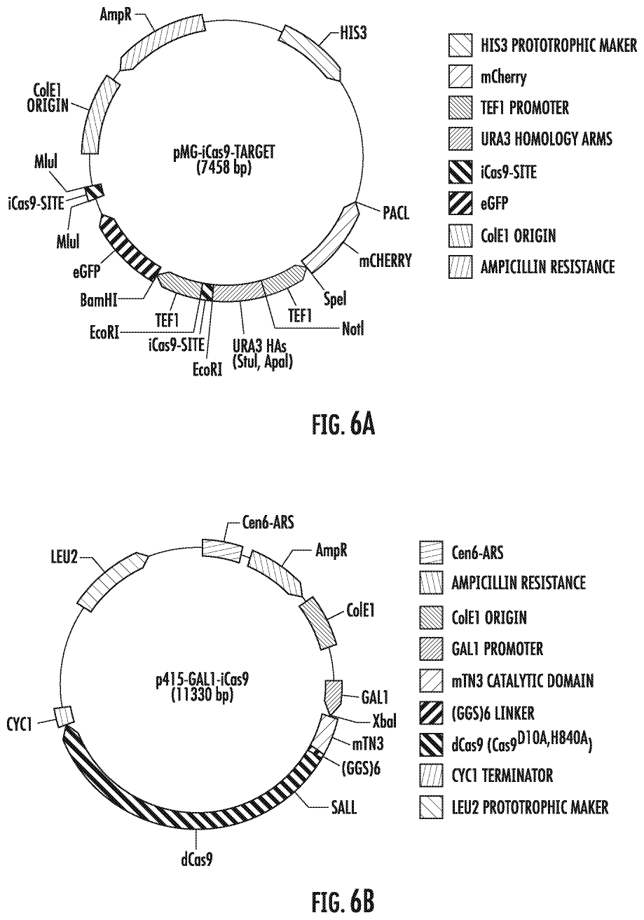

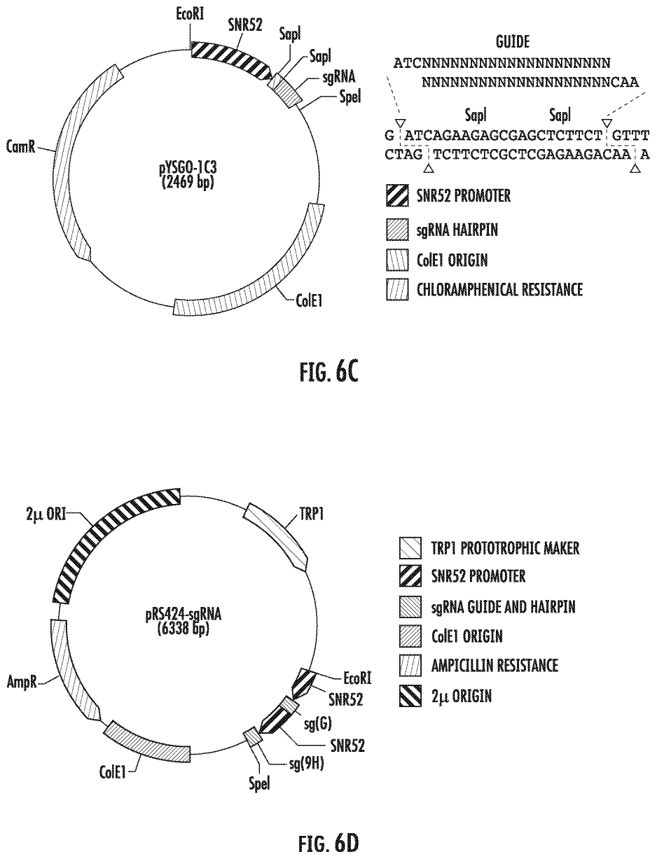

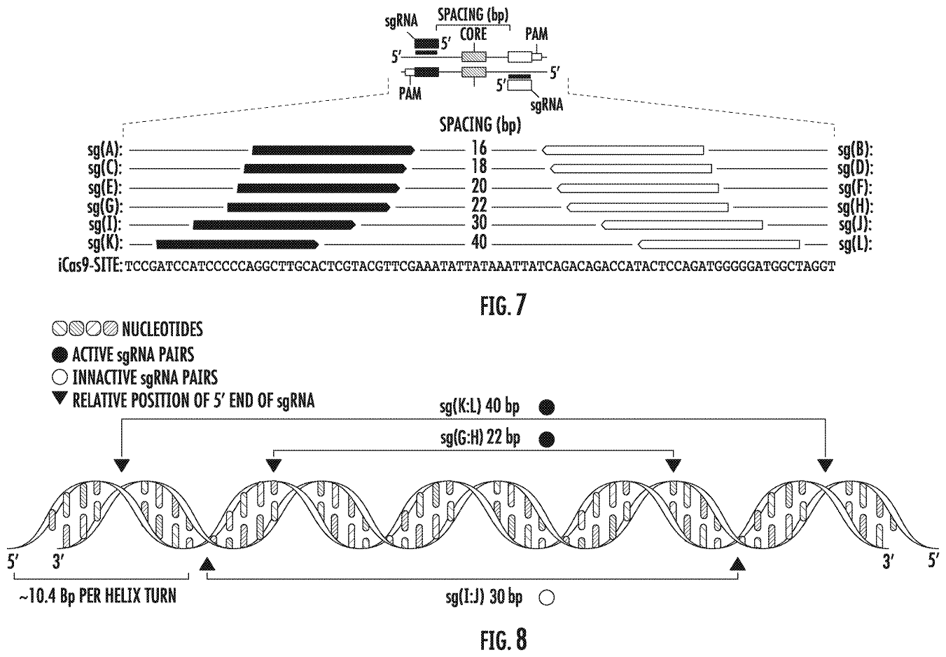

To develop an iCas9 capable of targeting eukaryotic genomic DNA, a yeast-based fluorescent reporter system was used to detect recombination. A Saccharomyces cerevisiae dual-fluorescent recombination reporter system, which contains GFP and mCherry expression cassettes was constructed and enabled detection of recombination using flow cytometry and fluorescence microscopy. Both GFP and mCherry were constitutively expressed from translation elongation factor 1 (Tef1) promoters. GFP was flanked by TN3 Res1 core sequences and resulted in GFP deletion upon iCas9 targeting. (FIG. 2A and FIGS. 6A-6D). Each core sequence was flanked with numerous PAMs, which enabled systematic analysis of sgRNA spacings (FIG. 7). iCas9 was placed on a yeast Cen6 vector with galactose inducible promoter and sgRNAs were placed on a yeast 2p vector with SNR52 promoters (FIGS. 6A-6D). Co-expression of iCas9 along with targeting sgRNA pairs resulted in loss-of-GFP detectable by flow cytometry (FIG. 2B). Single targeting with sgRNAs did not result in marked GFP-deletion (FIG. 2C). The observed requirement of cooperative targeting by sgRNAs matches mTN3's dimerization dependent function. sgRNA spacing's from 16 bp to 40 bp were analyzed. Symmetric spacing's of 22 bp and 40 bp were functional and resulted in 6.4±0.4% and 6.9±0.6% GFP-deletion respectively. However, 30 bp spacing symmetrically placed around the core sequence remained relatively non-functional while asymmetric spacing's of 31 bp around the core are functional (FIG. 2C). The observed functional spacing's are consistent with the requirement for targeting resolvase monomers to the same DNA helical face (See FIG. 8).

To confirm loss-of-GFP was due to GFP-deletion and not the result of spurious cell death or non-specific recombination, fluorescence microscopy was used to detect GFP and mCherry expression. All cells with a non-target guide, sg(−), expressed both GFP and mCherry. However, cooperative targeting with sgRNA pairs resulted in GFP-negative cells with intact mCherry expression (FIG. 2D). Recombination occurred on the DNA level by PCR with primers flanking the GFP and mCherry expression cassettes. The starting reporter resulted in a 5 Kb PCR product GFP-deletion generated a 4 Kb amplicon. The deletion product formed when iCas9 was co-expressed with sgRNA pairs, sg(G:H); however, no deletion product formed when iCas9 was co-expressed with sg(−). This indicates iCas9 targets DNA-deletion and its function is dependent on RNA-guidance (FIG. 2E). DSB-targeted DNA-deletion result in indel mutations. However, iCas9-mediated DNA-deletion should be free of mutations. To further characterize deletion products, the 4 Kb deletion amplicons were isolated, sub-cloned, and Sanger sequenced, and no indel mutations within the recombination product was observed (FIG. 2F). This further suggests the utility of iCas9 in mediating error-free DNA recombination.

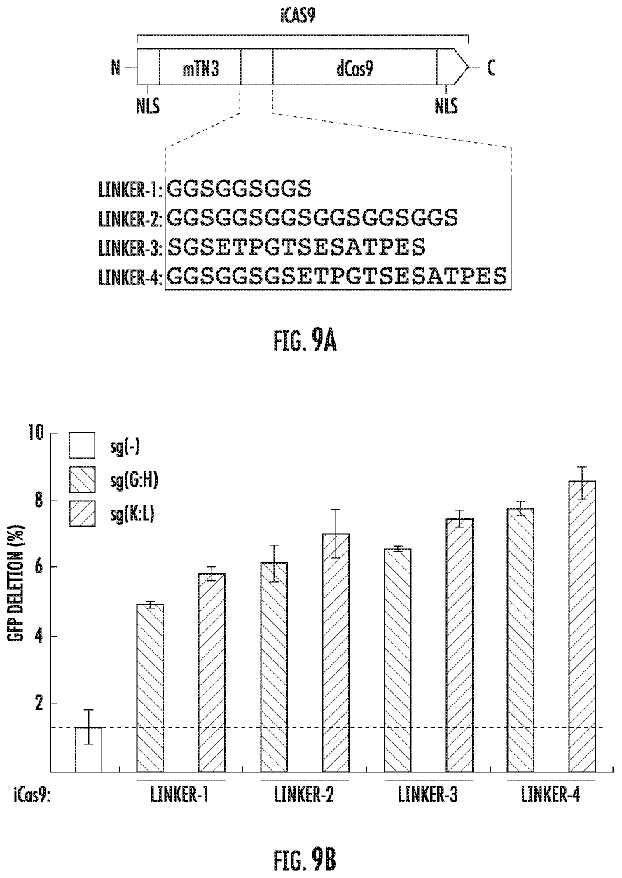

Aiming to improve iCas9 function, the effect of interdomain linker amino acid sequences was tested. These sequences included a range of flexible glycine serine and rigid linkers. Linker-3 was a common and effective linker used with Cas9 heterologous fusion proteins. Only subtle preference was observed for longer linker domains; however, these do not result in vivid improvement of iCas9 function (FIG. 9B). Henceforth, mTN3-(GGS)×6-dCas9 was used for further studies and referred to herein as “iCas9,” as its function has been extensively characterized in the yeast-based assays.

### 4. Validation in Human Cells

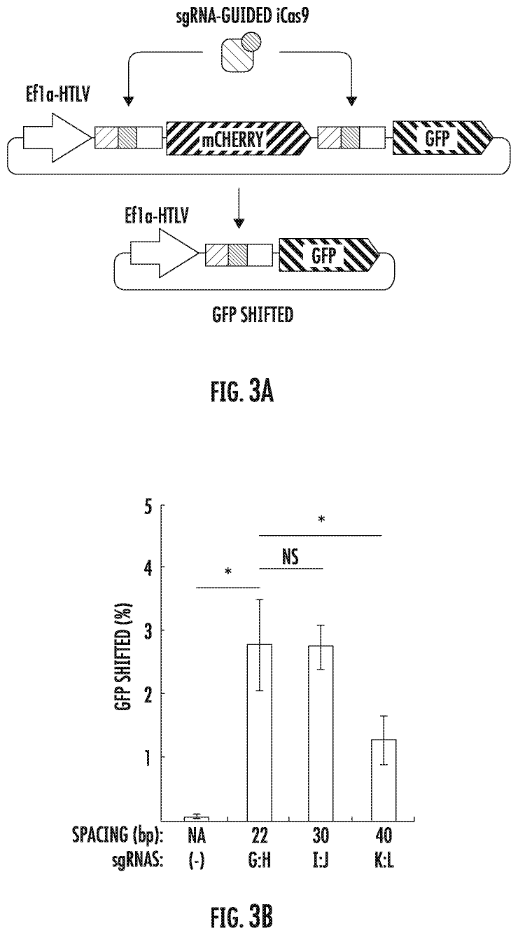

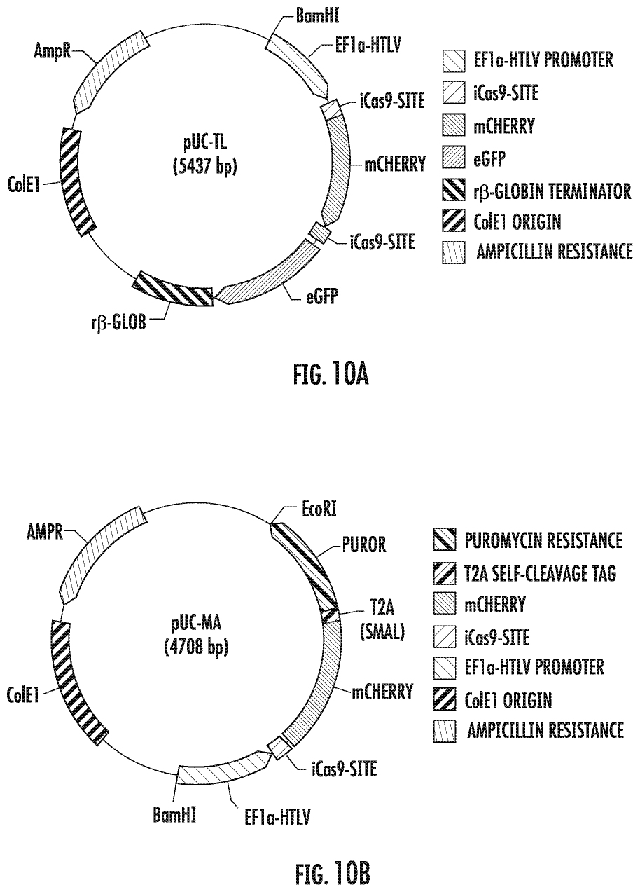

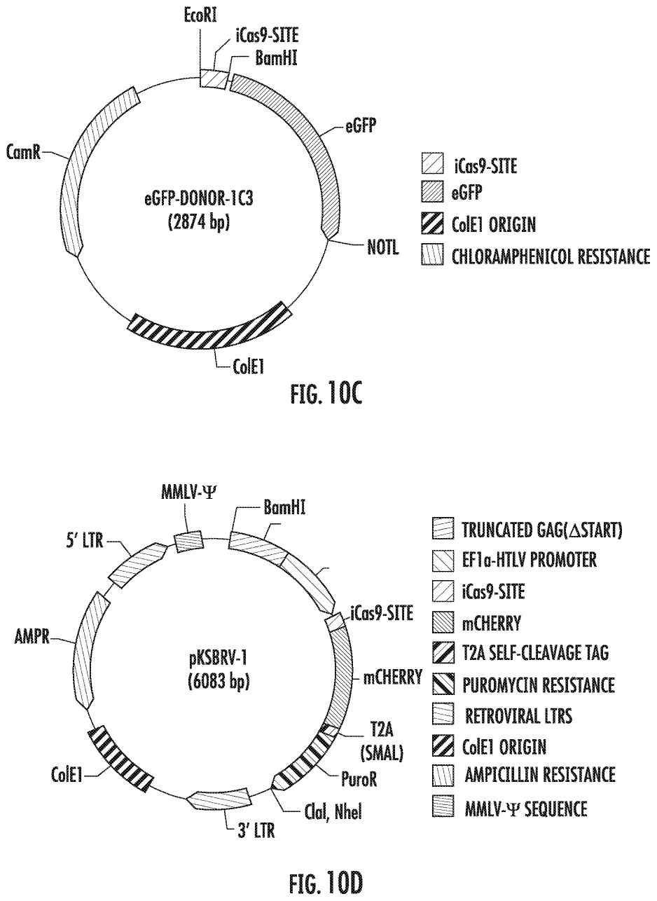

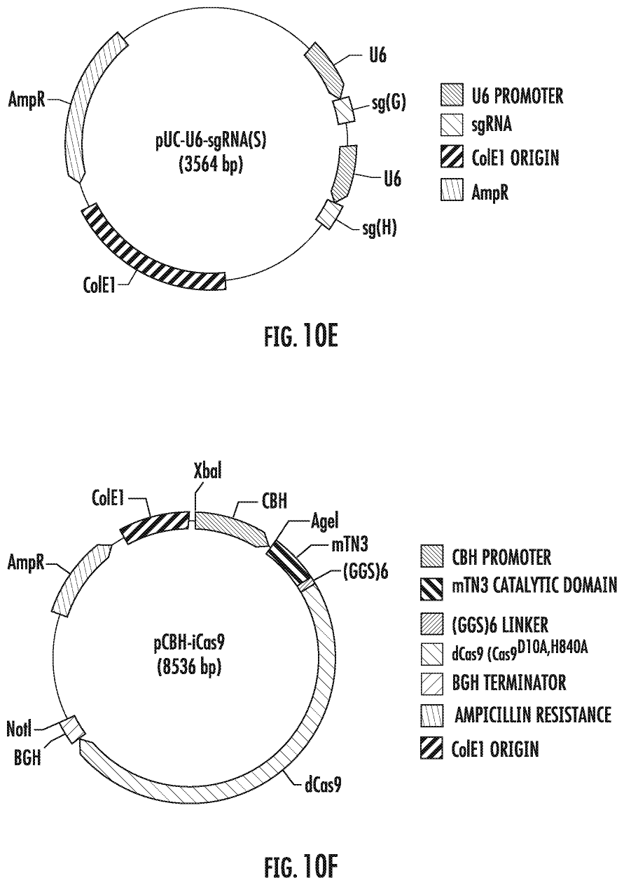

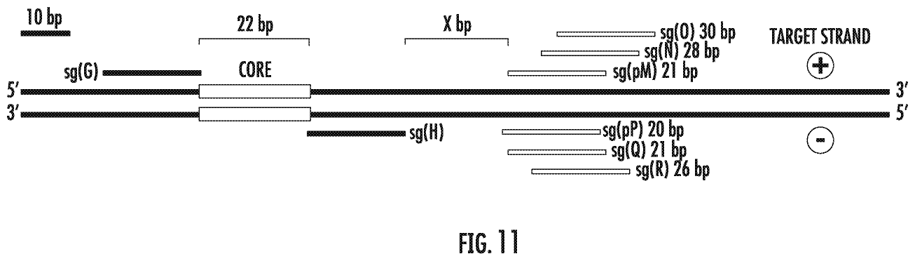

To assess the function of iCas9 in human cells, a dual-fluorescence detection plasmid-based reporter was developed. The reporter plasmid contained mCherry flanked by core recognition sites with GFP downstream (FIG. 3A, FIG. 10A). Therefore, mCherry deletion should result in cells expressing GFP only. Under this scenario, GFP expression remains relatively constant, while mCherry levels go to zero, yielding a population of cells with GFP levels shifted over mCherry. HEK293T cells was co-transfected with dual-reporter, sgRNA and iCas9 expression vectors while gating out untransfected cells. The shift of cells with GFP over mCherry expression was quantified using flow cytometry and analyzed to evaluate sgRNA spacings for our plasmid targeting assay. Interestingly 22, 30 and 40 bp shifted GFP expression, while a non-target guide, sg(−), resulted in no GFP shift. These results indicated both 22 and 30 bp are comparably functional when targeting plasmid substrates (FIG. 3B). Previous work with Gin-dCas9 fusions have reported the ability for 30 bp sgRNA spacing to target DNA deletion on plasmid substrates. This may be due to the use of supercoiled plasmids as substrates, which may support less stringent spacing requirements due to DNA coiling and 3D presentation. Nevertheless, 22 bp remained a highly functional sgRNA spacing and henceforth used since it is active in both plasmid and genomic assays.

Next to determine iCas9's ability to target intermolecular recombination, a two-plasmid reporter system for plasmid-to-plasmid integration was developed. One plasmid contains an elongation factor 1α (EF1α) human T-cell leukemia virus (HTLV) hybrid promoter, and a core target site upstream of a mCherry coding region. A second promoterless GFP-donor plasmid contains a core target sequence upstream of a GFP reading frame (FIGS. 10B and 10C). The GFP-donor plasmid conditionally expressed upon integration downstream of the EF1α-HTLV promoter resulted in dual-GFP and mCherry positive cells (FIG. 4A). GFP expression as detected by flow cytometry and fluorescence microscopy was used as an indicator of recombination efficiency. Co-transfection of iCas9 and a non-target guide control resulted in only mCherry expressing cells, however, targeting with sgRNAs at a 22 bp spacing resulted in GFP-positive cells (FIG. 4B). Flow cytometry measurements confirm the generation of mCherry-GFP dual-positive cells when targeting iCas9 with sg(G:H) (FIGS. 4C and 4D).

To determine if iCas9 can mediate plasmid-to-genome integration, the plasmid-based assay was adapted to detect genome integration (FIG. 5A). To accomplish this, the mCherry acceptor cassette was placed on a retroviral vector (FIG. 10D). HEK293Ts were transduced with viral particles containing the ‘acceptor-cassette’. This generated a population of cells with the mCherry acceptor cassette integrated into the genome. HEK293Ts were then transfected cells with iCas9, sgRNA(s) and GFP-Donor vector. In the first attempts, no increase in GFP+ cells in sg(G:H) were observed over a control guide, sg(−) (FIG. 5C). Even with validated plasmid-to-plasmid recombination, when the same ‘acceptor’ sequence is placed in the genome, no recombination was observed. iCas9 was verified to be capable of targeting both donor and acceptor sequences (FIG. 4); however this did not result in genome integration. This may be due to the inability of iCas9-bound GFP-donor plasmids to interact with the genomic acceptor locus.

Given iCas9's ability to mediate plasmid-to-plasmid but not plasmid-to-genome recombination, cooperative targeting may be necessary to enable genomic integration. Bacterial TN3 resolvase uses cooperative binding at accessory sites to ensure efficient recombination of cointegrate products, where TN3 resolvase coordinates substrate DNA bending, supercoiling and 3D positioning. Multiplex sgRNAs targeting can recreate accessory site binding, which should allow for extra mTN3 domains to coordinate interaction between GFP-donor and the acceptor locus. To test this, a series of sgRNAs adjacent to the target core sites were designed. These sgRNAs were targeted to either the ‘+’ or ‘−’ strand at varying base pair distances from the core target site (FIG. 5B, Supplemental FIG. 6). These accessory guides were co-transfected with sg(G:H), GFP-donor and iCas9 into the mCherry-acceptor line. A 10-fold increase in the number of GFP+ cells over the control guide was observed when targeting with accessory sg(M) (FIG. 5C). The recombination product was further characterized via PCR with primers flanking the integration junction. Integration of GFP into the acceptor locus was detected when targeting with sg(G), (H) and (M) (multiplex-targeting) (FIG. 5D). To further confirm the identity of this amplicon, the recombination product was subcloned and sequenced. Importantly, sequencing indicated the recombination product was free of unwanted indel mutations (FIG. 5E). On the other hand, targeting DNA integration using DSBs created by wildtype Cas9 induced indel mutations (FIG. 5F), which could be detrimental for many downstream applications.

### 5. Sequences Used

Table 1 lists the sgRNA guide sequences, and Table 2 lists the primers and oligonucleotides used.

TABLE 1

| Host: | Letter: | Guide Sequence: | Note: | SEQ ID NO. |
|---|---|---|---|---|
| Yeast | (—) | AGAAGAGCGAGCTCTTCT | Control, non-target | 3 |
| Yeast | A | CGAACGTACGAGTGCAAGCC | 16 bp spacing left | 4 |
| Yeast | C | GAACGTACGAGTGCAAGCCT | 18 bp spacing left | 5 |
| Yeast | E | AACGTACGAGTGCAAGCCTG | 20 bp spacing left | 6 |
| Yeast | G | ACGTACGAGTGCAAGCCTGG | 22 bp spacing left | 7 |
| Yeast | I | ACGAGTGCAAGCCTGGGGGA | 30 bp spacing left | 8 |
| Yeast | K | TGCAAGCCTGGGGGATGGAT | 40 bp spacing left | 9 |
| Yeast | B | CAGACAGACCATACTCCAGA | 16 bp spacing right | 10 |
| Yeast | D | AGACAGACCATACTCCAGAT | 18 bp spacing right | 11 |
| Yeast | F | GACAGACCATACTCCAGATG | 20 bp spacing right | 12 |
| Yeast | H | ACAGACCATACTCCAGATGG | 22 bp spacing right | 13 |
| Yeast | J | ACCATACTCCAGATGGGGGA | 30 bp spacing right | 14 |
| Yeast | L | ACTCCAGATGGGGGATGGCT | 40 bp spacing right | 15 |
| Human | (—) | GGGTCTTCGAGAAGACCT | Control, non-target | 16 |
| Human | G | GACGTACGAGTGCAAGCCTGG | 22 bp spacing left | 17 |
| Human | H | GACAGACCTTACTCCAGAAGG | 22 bp spacing right | 18 |
| Human | K | GTGCAAGCCTGGGGGAAGGAT | 40 bp spacing left | 19 |
| Human | L | GACTCCAGAAGGGGGAAGGCT | 40 bp spacing right | 20 |
| Human | I | GACGAGTGCAAGCCTGGGGGA | 30 bp spacing left | 21 |
| Human | J | GACCTTACTCCAGAAGGGGGA | 30 bp spacing right | 22 |
| Human | M | GTTGCTCACCATGGTGGCGAC | Accessory, +21 bp | 23 |
| Human | N | GCTCGCCCTTGCTCACCATGG | Accessory, +28 bp | 24 |
| Human | O | GCTCCTCGCCCTTGCTCACCA | Accessory, +31 bp | 25 |
| Human | P | GGTCGCCACCATGGTGAGCA | Accessory, −20 bp | 26 |
| Human | Q | GTCGCCACCATGGTGAGCAA | Accessory, −21 bp | 27 |
| Human | R | GCACCATGGTGAGCAAGGGCG | Accessory, −26 bp | 28 |

TABLE 2

| Primer | Sequence | Note: | SEQ ID NO. |
|---|---|---|---|
| 1 | CGCATATGTGGTGTTGAAGA | Yeast URA3 PCR F | 29 |
| 2 | CTAGGGCTTTCTGCTCTGTCAT | Yeast HIS3 PCR R | 30 |
| 3 | TGGAGGGCACAGTTAAGCCG | Yeast URA3 PCR F-2 | 31 |
| 4 | AATACCGCCTTTGAGTGAGC | Standard vector PCR/Seq. R | 32 |
| 5 | AGCTGTGACCGGCGCCTACG | Human EF1α-HTLV PCR F | 33 |
| 6 | CTGAGCACCCAGTCCGCCCTGAG | Human eGFP R | 34 |
| 7 | AATTCTCCGATCCATCCCCCAGGCTTG | Yeast iCas9-site EcoRI-end | 35 |
| | CACTCGTACGTTCGAAATAT | Top 1 | |
| 8 | ATAATATTTCGAACGTACGAGTGCAA | Yeast iCas9-site EcoRI-end | 36 |
| | GCCTGGGGGATGGATCGGAG | Bottom 1 | |
| 9 | TATAAATTATCAGACAGACCATACTC | Yeast iCas9-site EcoRI-end | 37 |
| | CAGATGGGGGATGGCTAGGTG | Top 2 | |
| 10 | AATTCACCTAGCCATCCCCCATCTGGA | Yeast iCas9-site EcoRI-end | 38 |
| | GTATGGTCTGTCTGATAATTT | Bottom 2 | |
| 11 | CGCGTTCCGATCCATCCCCCAGGCTTG | Yeast iCas9-site MluI-end | 39 |
| | CACTCGTACGTTCGAAATAT | Top 1 | |
| 12 | ATAATATTTCGAACGTACGAGTGCAA | Yeast iCas9-site MluI-end | 40 |
| | GCCTGGGGGATGGATCGGAA | Bottom 1 | |
| 13 | TATAAATTATCAGACAGACCATACTC | Yeast iCas9-site Mlu-endI | 41 |
| | CAGATGGGGGATGGCTAGGTA | Top 2 | |
| 14 | CGCGTACCTAGCCATCCCCCATCTGG | Yeast iCas9-site MluI-end | 42 |
| | AGTATGGTCTGTCTGATAATTT | Bottom 2 | |
| 15 | AATTCTCCGATCCTTCCCCCAGGCTTG | Human iCas9-site EcoRI- | 43 |
| | CACTCGTACGTTCGAAATAT | end Top 1 | |
| 16 | CTCCGATCCTTCCCCCAGGCTTGCACT | Human iCas9-site Blunt-end | 44 |
| | CGTACGTTCGAAATAT | Top 1 | |
| 17 | ATAATATTTCGAACGTACGAGTGCAA | Human iCas9-site Bottom 1 | 45 |
| | GCCTGGGGGAAGGATCGGAG | | |
| 18 | TATAAATTATCAGACAGACCTTACTCC | Human iCas9-site Top 1 | 46 |
| | AGAAGGGGGAAGGCTAGGTG | | |
| 19 | GATCCACCTAGCCTTCCCCCTTCTGGA | Human iCas9-site BamHI- | 47 |
| | GTAAGGTCTGTCTGATAATTT | end Bottom 2 | |
| 20 | CACCTAGCCTTCCCCCTTCTGGAGTAA | Human iCas9-site Blunt-end | 48 |
| | GGTCTGTCTGATAATTT | Bottom 2 | |
| 21 | AGAACAGTTGATAGAGGAGGGAGCG | Linker-1 Oligo Top | 49 |
| | GGGGAAGCGGTGGCTCA | | |
| 22 | CATTGAGCCACCGCTTCCCCCGCTCCC | Linker-1 Oligo Bottom | 50 |
| | TCCTCTATCAACTGT | | |
| 23 | AGAACTGTTGACCGAGGTGGTTCAGG | Linker-2 Oligo Top 1 | 51 |
| | AGGAAGTGGA | | |
| 24 | ACCTCCACTTCCTCCTGAACCACCTCG | Linker-2 Oligo Bottom 1 | 52 |
| | GTCAACAGT | | |
| 25 | GGTTCAGGGGGAAGTGGTGGCTCCGG | Linker-2 Oligo Top 2 | 53 |
| | TGGGTCT | | |
| 26 | CATAGACCCACCGGAGCCACCACTTC | Linker-2 Oligo Bottom 2 | 54 |
| | CCCCTGA | | |
| 27 | AGAACAGTTGATCGGAGCGGTTCTGA | Linker-3 Oligo Top 1 | 55 |
| | GACT | | |
| 28 | CGGAGTCTCAGAACCGCTCCGATCAA | Linker-3 Oligo Bottom 1 | 56 |
| | CTGT | | |
| 29 | CCGGGAACCTCAGAGTCTGCTACGCC | Linker-3 Oligo Top 2 | 57 |
| | GGAAAGC | | |
| 30 | CATGCTTTCCGGCGTAGCAGACTCTG | Linker-3 Oligo Bottom 2 | 58 |
| | AGGTTCC | | |
| 31 | AGAACCGTAGATCGCGGGGGCTCTGG | Linker-4 Oligo Top 1 | 59 |
| | AGGATCAGGTA | | |
| 32 | CGCTACCTGATCCTCCAGAGCCCCCG | Linker-4 Oligo Bottom 1 | 60 |
| | CGATCTACGGT | | |
| 33 | GCGAAACGCCGGGTACTAGCGAAAGC | Linker-4 Oligo Top 2 | 61 |
| | GCGACACCTGAGAGT | | |
| 34 | CATACTCTCAGGTGTCGCGCTTTCGCT | Linker-4 Oligo Bottom 2 | 62 |
| | AGTACCCGGCGTTT | | |
| 35 | ACGGCTCTTCGATGCCCAAAAAGAAG | mTN3 N-terminus | 63 |
| | AGGAAAGT | | |
| 36 | AGCGCTCTTCATCTGTCTACAGTCCTC | mTN3 Catalytic domain R, | 64 |
| | CTGCG | SapI | |
| 37 | ACGGCTCTTCGCATTTTTTTCCCGGGG | Cas9 N-terminus R SapI | 65 |
| | GATCC | | |
| 38 | GCGCTCTTCAGCCATCGGCACAAACA | Cas9 D10A, F SapI | 66 |
| | GCG | | |
| 39 | ACGGCTCTTCGGGCGAGCCCAATGGA | Cas9 D10A, R SapI | 67 |
| | GTACTTCTT | | |
| 40 | GCGCTCTTCAGCCATCGTGCCCCAGTC | Cas9 H840A F SapI | 68 |
| | TTTT | | |
| 41 | ACGGCTCTTCGGGCATCCACGTCGTA | Cas9 H840A R SapI | 69 |
| | GTCGGAG | | |
| 42 | ATACACCGGTGCCACCATGCCCAAAA | iCas9 F, AgeI | 70 |
| | AGAAGAGGAAAGT | | |
| 43 | GATGACAATTGTCACACCTTCCTCTTC | iCas9 R, MfeI | 71 |
| | TTCTTG | | |
| 44 | GTGAGAATTCTCTTTGAAAAGATAAT | Yeast sgRNA cassette F, | 72 |
| | GTATGATTATGC | EcoRI | |
| 45 | ACGGCTCTTCGTCTTTGAAAAGATAAT | Yeast sgRNA cassette R, | 73 |
| | GTATGATTATGC | SapI | |
| 46 | ACGGCTCTTCGAGAGTCTCCAATTATC | Yeast sgRNA cassette F, | 74 |
| | TAGTAAAAAAAGCACC | SapI | |
| 47 | CGTCATGTCACTAGTAGAGTCTCCAAT | Yeast sgRNA cassette R, | 75 |
| | TATCTAGTAAAAAAAGCACC | SpeI | |
| 48 | GTGAGAATTCGAGGGCCTATTTCCCA | Human sgRNA cassette F, | 76 |
| | TGAT | EcoRI | |
| 49 | ACGGCTCTTCGTCTGTCTGCAGAATTG | Human sgRNA cassette R, | 77 |
| | GCG | SapI | |
| 50 | AGCGCTCTTCTAGAGAGGGCCTATTTC | Human sgRNA cassette F, | 78 |
| | CCATGAT | SapI | |
| 51 | CGTCATGTCTCTAGATTTGTCTGCAGA | Human sgRNA cassette R, | 79 |
| | ATTGGCG | SpeI | |
| 52 | ATCCGAACGTACGAGTGCAAGCC | Yeast sg(A) Top | 80 |
| 53 | AACGGCTTGCACTCGTACGTTCG | Yeast sg(A) Bottom | 81 |
| 54 | ATCGAACGTACGAGTGCAAGCCT | Yeast sg(C) Top | 82 |
| 55 | AACAGGCTTGCACTCGTACGTTC | Yeast sg(C) Bottom | 83 |
| 56 | ATCAACGTACGAGTGCAAGCCTG | Yeast sg(E) Top | 84 |
| 57 | AACCAGGCTTGCACTCGTACGTT | Yeast sg(E) Bottom | 85 |
| 58 | ATCACGTACGAGTGCAAGCCTGG | Yeast sg(G) Top | 86 |
| 59 | AACCCAGGCTTGCACTCGTACGT | Yeast sg(G) Bottom | 87 |
| 60 | ATCACGAGTGCAAGCCTGGGGGA | Yeast sg(I) Top | 88 |
| 61 | AACTCCCCCAGGCTTGCACTCGT | Yeast sg(I) Bottom | 89 |
| 62 | ATCTGCAAGCCTGGGGGATGGAT | Yeast sg(K) Top | 90 |
| 63 | AACATCCATCCCCCAGGCTTGCA | Yeast sg(K) Bottom | 91 |
| 64 | ATCCAGACAGACCATACTCCAGA | Yeast sg(B) Top | 92 |
| 65 | AACTCTGGAGTATGGTCTGTCTG | Yeast sg(B) Bottom | 93 |
| 66 | ATCAGACAGACCATACTCCAGAT | Yeast sg(D) Top | 94 |
| 67 | AACATCTGGAGTATGGTCTGTCT | Yeast sg(D) Bottom | 95 |
| 68 | ATCGACAGACCATACTCCAGATG | Yeast sg(F) Top | 96 |
| 69 | AACCATCTGGAGTATGGTCTGTC | Yeast sg(F) Bottom | 97 |
| 70 | ATCACAGACCATACTCCAGATGG | Yeast sg(H) Top | 98 |
| 71 | AACCCATCTGGAGTATGGTCTGT | Yeast sg(H) Bottom | 99 |
| 72 | ATCACCATACTCCAGATGGGGGA | Yeast sg(J) Top | 100 |
| 73 | AACTCCCCCATCTGGAGTATGGT | Yeast sg(J) Bottom | 101 |
| 74 | ATCACTCCAGATGGGGGATGGCT | Yeast sg(L) Top | 102 |
| 75 | AACAGCCATCCCCCATCTGGAGT | Yeast sg(L) Bottom | 103 |
| 76 | CACCGACGTACGAGTGCAAGCCTGG | Human sg(G) Top | 104 |
| 77 | AAACCCAGGCTTGCACTCGTACGTC | Human sg(G) Bottom | 105 |
| 78 | CACCGACAGACCTTACTCCAGAAGG | Human sg(H) Top | 106 |
| 79 | AAACCCTTCTGGAGTAAGGTCTGTC | Human sg(H) Bottom | 107 |
| 80 | CACCGTGCAAGCCTGGGGGAAGGAT | Human sg(K) Top | 108 |
| 81 | AAACATCCTTCCCCCAGGCTTGCAC | Human sg(K) Bottom | 109 |
| 82 | CACCGACTCCAGAAGGGGGAAGGCT | Human sg(L) Top | 110 |
| 83 | AAACAGCCTTCCCCCTTCTGGAGTC | Human sg(L) Bottom | 111 |
| 84 | CACCGACGAGTGCAAGCCTGGGGGA | Human sg(I) Top | 112 |
| 85 | AAACTCCCCCAGGCTTGCACTCGTC | Human sg(I) Top | 113 |
| 86 | CACCGACCTTACTCCAGAAGGGGGA | Human sg(J) Top | 114 |
| 87 | AAACTCCCCCTTCTGGAGTAAGGTC | Human sg(J) Bottom | 115 |

The nucleic acid sequences for the exemplary guide sequences are listed below set forth in SEQ IN NOs. 116-119. The nucleic acid sequence and the amino acid sequence of an exemplary Cas9 fusion protein are listed below and set forth in SEQ ID NOs. 2 and 3.

iCas9-site (Yeast) (88 bp) (SEQ ID NO. 116): sg(G:H) underlined, PAM/s bolded, TN3 Res1 sequence italicized

TCCGATCCATCCCCCAGGCTTGCACTCGTACGTTCGAAATATTATAAATT
ATCAGACAGACCATACTCCAGATGGGGGATGGCTAGGT

iCas9-site (Human) (88 bp) (SEQ ID NO. 117): sg(G:H) underlined, PAMs bolded, TN3 Res1 sequence italicized

TCCGATCCTTCCCCCAGGCTTGCACTCGTACGTTCGAAATATTATAAATT
ATCAGACAGACCTTACTCCAGAAGGGGGAAGGCTAGGT

iCas9-site (Human) with Accessory Targets (123 bp) (SEQ ID NO. 118): sg(G:H) and sg(M) underlined, PAMs bolded, TN3 Res1 sequence italicized

TCCGATCCTTCCCCCAGGCTTGCACTCGTACGTTCGAAATATTATAAATT
ATCAGACAGACCTTACTCCAGAAGGGGGAAGGCTAGGTGGCTACCGGTCG
CCACCATGGTGAGCAAGGGCGAG

iCas9-site (Human) with Accessory Targets (123 bp) (SEQ ID NO. 119): sg(G:H) and sg(N) underlined, PAMs bolded, TN3 Res1 sequence italicized

TCCGATCCTTCCCCCAGGCTTGCACTCGTACGTTCGAAATATTATAAATT
ATCAGACAGACCTTACTCCAGAAGGGGGAAGGCTAGGTGGCTACCGGTCG
CCACCATGGTGAGCAAGGGCGAG

iCas9 Amino Acid Sequence (NLS-GGS-mTN3-GGS*6-dCas9-NLS) (1556 aa) (SEQ ID NO. 1): SV40 NLS underlined, mTN3 Catalytic Domain (TN3-TnpR G70S, D102Y, E124Q) bolded, GGS*6 Interdomain Linker italicized, dCas9 (Cas9 D10A, H840A) without modifications

MPKKKRKVGGSMRIFGYARVSTSQQSLDIQIRALKDAGVKANRIFTDKAS
GSSTDREGLDLLRMKVEEGDVILVKKLDRLSRDTADMIQLIKEFDAQGVA
VRFIDDGISTDGYMGQMVVTILSAVAQAERRRILQRTNEGRQEAKLKGIK
FGRRRTVDRGGSGGSGGSGGSGGSGGSMDKKYSIGLAIGTNSVGWAVITD
EYKVPSKKFKVLGNTDRHSIKKNLIGALLFDSGETAEATRLKRTARRRYT
RRKNRICYLQEIFSNEMAKVDDSFFHRLEESFLVEEDKKHERHPIFGNIV
DEVAYHEKYPTIYHLRKKLVDSTDKADLRLIYLALAHMIKFRGHFLIEGD
LNPDNSDVDKLFIQLVQTYNQLFEENPINASGVDAKAILSARLSKSRRLE
NLIAQLPGEKKNGLFGNLIALSLGLTPNFKSNFDLAEDAKLQLSKDTYDD
DLDNLLAQIGDQYADLFLAAKNLSDAILLSDILRVNTEITKAPLSASMIK
RYDEHHQDLTLLKALVRQQLPEKYKEIFFDQSKNGYAGYIDGGASQEEFY
KFIKPILEKMDGTEELLVKLNREDLLRKQRTFDNGSIPHQIHLGELHAIL
RRQEDFYPFLKDNREKIEKILTFRIPYYVGPLARGNSRFAWMTRKSEETI
TPWNFEEVVDKGASAQSFIERMTNFDKNLPNEKVLPKHSLLYEYFTVYNE
LTKVKYVTEGMRKPAFLSGEQKKAIVDLLFKTNRKVTVKQLKEDYFKKIE
CFDSVEISGVEDRFNASLGTYHDLLKIIKDKDFLDNEENEDILEDIVLTL
TLFEDREMIEERLKTYAHLFDDKVMKQLKRRRYTGWGRLSRKLINGIRDK
QSGKTILDFLKSDGFANRNFMQLIHDDSLTFKEDIQKAQVSGQGDSLHEH
IANLAGSPAIKKGILQTVKVVDELVKVMGRHKPENIVIEMARENQTTQKG
QKNSRERMKRIEEGIKELGSQILKEHPVENTQLQNEKLYLYYLQNGRDMY
VDQELDINRLSDYDVDAIVPQSFLKDDSIDNKVLTRSDKNRGKSDNVPSE
EVVKKMKNYWRQLLNAKLITQRKFDNLTKAERGGLSELDKAGFIKRQLVE
TRQITKHVAQILDSRMNTKYDENDKLIREVKVITLKSKLVSDFRKDFQFY
KVREINNYHHAHDAYLNAVVGTALIKKYPKLESEFVYGDYKVYDVRKMIA
KSEQEIGKATAKYFFYSNIMNFFKTEITLANGEIRKRPLIETNGETGEIV
WDKGRDFATVRKVLSMPQVNIVKKTEVQTGGFSKESILPKRNSDKLIARK
KDWDPKKYGGFDSPTVAYSVLVVAKVEKGKSKKLKSVKELLGITIMERSS
FEKNPIDFLEAKGYKEVKKDLIIKLPKYSLFELENGRKRMLASAGELQKG
NELALPSKYVNFLYLASHYEKLKGSPEDNEQKQLFVEQHKHYLDEIIEQI
SEFSKRVILADANLDKVLSAYNKHRDKPIREQAENIIHLFTLTNLGAPAA
FKYFDTTIDRKRYTSTKEVLDATLIHQSITGLYETRIDLSQLGGDSRADP
KKKRKV

### REFERENCES

(1) Cong, L.; Ran, F. A.; Cox, D.; Lin, S.; Barretto, R.; Habib, N.; Hsu, P. D.; Wu, X.; Jiang, W.; Marraffini, L. A.; et al. Multiplex Genome Engineering Using CRISPR/Cas Systems. Science 2013, 339 (6121), 819-823. https://doi.org/10.1126/science.1231143.
(2) Mali, P.; Yang, L.; Esvelt, K. M.; Aach, J.; Guell, M.; DiCarlo, J. E.; Norville, J. E.; Church, G. M. RNA-Guided Human Genome Engineering via Cas9. Science 2013, 339 (6121), 823-826. https://doi.org/10.1126/science.1232033.
(3) Zetsche, B.; Gootenberg, J. S.; Abudayyeh, O. O.; Slaymaker, I. M.; Makarova, K. S.; Essletzbichler, P.; Volz, S. E.; Joung, J.; van der Oost, J.; Regev, A.; et al. Cpf1 Is a Single RNA-Guided Endonuclease of a Class 2 CRISPR-Cas System. Cell 2015, 163 (3), 759-771. https://doi.org/10.1016/j.cell.2015.09.038.
(4) Sander, J. D.; Joung, J. K. CRISPR-Cas Systems for Editing, Regulating and Targeting Genomes. Nat. Biotechnol. 2014, 32 (4), 347-355. https://doi.org/10.1038/nbt.2842.
(5) Brookhouser, N.; Raman, S.; Potts, C.; Brafman, D. A. May I Cut in? Gene Editing Approaches in Human Induced Pluripotent Stem Cells. Cells 2017, 6 (1). https://doi.org/10.3390/cells6010005.
(6) Suzuki, K.; Tsunekawa, Y.; Hernandez-Benitez, R.; Wu, J.; Zhu, J.; Kim, E. J.; Hatanaka, F.; Yamamoto, M.; Araoka, T.; Li, Z.; et al. In Vivo Genome Editing via CRISPR/Cas9 Mediated Homology-Independent Targeted Integration. Nature 2016, 540 (7631), 144-149. https://doi.org/10.1038/nature20565.
(7) He, X.; Tan, C.; Wang, F.; Wang, Y.; Zhou, R.; Cui, D.; You, W.; Zhao, H.; Ren, J.; Feng, B. Knock-in of Large Reporter Genes in Human Cells via CRISPR/Cas9-Induced Homology-Dependent and Independent DNA Repair. Nucleic Acids Res. 2016, 44 (9), e85. https://doi.org/10.1093/nar/gkw064.
(8) Schmid-Burgk, J. L.; Höning, K.; Ebert, T. S.; Hornung, V. CRISPaint Allows Modular Base-Specific Gene Tagging Using a Ligase-4-Dependent Mechanism. Nat. Commun. 2016, 7, 12338. https://doi.org/10.1038/ncomms12338.
(9) Orthwein, A.; Noordermeer, S. M.; Wilson, M. D.; Landry, S.; Enchev, R. I.; Sherker, A.; Munro, M.; Pinder, J.; Salsman, J.; Dellaire, G.; et al. A Mechanism for the Suppression of Homologous Recombination in G1 Cells. Nature 2015, advance online publication. https://doi.org/10.1038/nature16142.
(10) Ihry, R. J.; Worringer, K. A.; Salick, M. R.; Frias, E.; Ho, D.; Theriault, K.; Kommineni, S.; Chen, J.; Sondey, M.; Ye, C.; et al. P53 Inhibits CRISPR-Cas9 Engineering in Human Pluripotent Stem Cells. Nat. Med. 2018, 24 (7), 939-946. https://doi.org/10.1038/s41591-018-0050-6.
(11) Haapaniemi, E.; Botla, S.; Persson, J.; Schmierer, B.; Taipale, J. CRISPR-Cas9 Genome Editing Induces a P53-Mediated DNA Damage Response. Nat. Med. 2018, 24 (7), 927. https://doi.org/10.1038/s41591-018-0049-z.
(12) Fu, Y.; Foden, J. A.; Khayter, C.; Maeder, M. L.; Reyon, D.; Joung, J. K.; Sander, J. D. High-Frequency off-Target Mutagenesis Induced by CRISPR-Cas Nucleases in Human Cells. Nat. Biotechnol. 2013, 31 (9), 822-826. https://doi.org/10.1038/nbt.2623.
(13) Kosicki, M.; Tomberg, K.; Bradley, A. Repair of Double-Strand Breaks Induced by CRISPR-Cas9 Leads to Large Deletions and Complex Rearrangements. Nat. Biotechnol. 2018, 36 (8), 765-771. https://doi.org/10.1038/nbt.4192.
(14) Komor, A. C.; Kim, Y. B.; Packer, M. S.; Zuris, J. A.; Liu, D. R. Programmable Editing of a Target Base in Genomic DNA without Double-Stranded DNA Cleavage. Nature 2016, 533 (7603), 420-424. https://doi.org/10.1038/nature17946.
(15) Komor, A. C.; Zhao, K. T.; Packer, M. S.; Gaudelli, N. M.; Waterbury, A. L.; Koblan, L. W.; Kim, Y. B.; Badran, A. H.; Liu, D. R. Improved Base Excision Repair Inhibition and Bacteriophage Mu Gam Protein Yields C:G-to-T:A Base Editors with Higher Efficiency and Product Purity. Sci. Adv. 2017, 3 (8), eaao4774. https://doi.org/10.1126/sciadv.aao4774.
(16) Gaj, T.; Sirk, S. J.; Barbas, C. F. Expanding the Scope of Site-Specific Recombinases for Genetic and Metabolic Engineering. Biotechnol. Bioeng. 2014, 111 (1), 1-15. https://doi.org/10.1002/bit.25096.
(17) Standage-Beier, K.; Wang, X. Genome Reprogramming for Synthetic Biology. Front. Chem. Sci. Eng. 2017, 11 (1), 37-45. https://doi.org/10.1007/s11705-017-1618-2.
(18) Grindley, N. D. F.; Whiteson, K. L.; Rice, P. A. Mechanisms of Site-Specific Recombination. Annu. Rev. Biochem. 2006, 75 (1), 567-605. https://doi.org/10.1146/annurev.biochem.73.011303.073908.
(19) Brafman, D.; Willert, K. Gene Transduction Approaches in Human Embryonic Stem Cells. Methodol. Adv. Cult. Manip. Util. Embryonic Stem Cells Basic Pract. Appl. 2011. https://doi.org/10.5772/14163.
(20) St-Pierre, F.; Cui, L.; Priest, D. G.; Endy, D.; Dodd, I. B.; Shearwin, K. E. One-Step Cloning and Chromosomal Integration of DNA. ACS Synth. Biol. 2013, 2 (9), 537-541. https://doi.org/10.1021/sb400021j.
(21) Karpinski, J.; Hauber, I.; Chemnitz, J.; Schafer, C.; Paszkowski-Rogacz, M.; Chakraborty, D.; Beschorner, N.; Hofmann-Sieber, H.; Lange, U. C.; Grundhoff, A.; et al. Directed Evolution of a Recombinase That Excises the Provirus of Most HIV-1 Primary Isolates with High Specificity. Nat. Biotechnol. 2016, 34 (4), 401-409. https://doi.org/10.1038/nbt.3467.
(22) Akopian, A.; He, J.; Boocock, M. R.; Stark, W. M. Chimeric Recombinases with Designed DNA Sequence Recognition. Proc. Natl. Acad. Sci. 2003, 100 (15), 8688-8691. https://doi.org/10.1073/pnas.1533177100.
(23) Mercer, A. C.; Gaj, T.; Fuller, R. P.; Barbas, C. F. Chimeric TALE Recombinases with Programmable DNA Sequence Specificity. Nucleic Acids Res. 2012, gks875. https://doi.org/10.1093/nar/gks875.
(24) Gordley, R. M.; Gersbach, C. A.; Barbas, C. F. Synthesis of Programmable Integrases. Proc. Natl. Acad. Sci. 2009, 106 (13), 5053-5058. https://doi.org/10.1073/pnas.0812502106.
(25) Arnold, P. H.; Blake, D. G.; Grindley, N. D.; Boocock, M. R.; Stark, W. M. Mutants of Tn3 Resolvase Which Do Not Require Accessory Binding Sites for Recombination Activity. EMBO J. 1999, 18 (5), 1407-1414. https://doi.org/10.1093/emboj/18.5.1407.
(26) Prorocic, M. M.; Wenlong, D.; Olorunniji, F. J.; Akopian, A.; Schloetel, J.-G.; Hannigan, A.; McPherson, A. L.; Stark, W. M. Zinc-Finger Recombinase Activities in Vitro. Nucleic Acids Res. 2011, 39 (21), 9316-9328. https://doi.org/10.1093/nar/gkr652.
(27) Yang, W.; Steitz, T. A. Crystal Structure of the Site-Specific Recombinase Gamma Delta Resolvase Complexed with a 34 Bp Cleavage Site. Cell 1995, 82 (2), 193-207.
(28) Li, W.; Kamtekar, S.; Xiong, Y.; Sarkis, G. J.; Grindley, N. D. F.; Steitz, T. A. Structure of a Synaptic Γδ Resolvase Tetramer Covalently Linked to Two Cleaved DNAs. Science 2005, 309 (5738), 1210-1215. https://doi.org/10.1126/science.1112064.
(29) Guilinger, J. P.; Thompson, D. B.; Liu, D. R. Fusion of Catalytically Inactive Cas9 to FokI Nuclease Improves the Specificity of Genome Modification. Nat. Biotechnol. 2014, 32 (6), 577-582. https://doi.org/10.1038/nbt.2909.
(30) Nishimasu, H.; Ran, F. A.; Hsu, P. D.; Konermann, S.; Shehata, S. I.; Dohmae, N.; Ishitani, R.; Zhang, F.; Nureki, O. Crystal Structure of Cas9 in Complex with Guide RNA and Target DNA. Cell 2014, 156 (5), 935-949. https://doi.org/10.1016/j.cell.2014.02.001.
(31) Standage-Beier, K.; Zhang, Q.; Wang, X. Targeted Large-Scale Deletion of Bacterial Genomes Using CRISPR-Nickases. ACS Synth. Biol. 2015, 4 (11), 1217-1225. https://doi.org/10.1021/acssynbio.5b00132.
(32) DiCarlo, J. E.; Norville, J. E.; Mali, P.; Rios, X.; Aach, J.; Church, G. M. Genome Engineering in Saccharomyces Cerevisiae Using CRISPR-Cas Systems. Nucleic Acids Res. 2013, 41 (7), 4336-4343. https://doi.org/10.1093/nar/gkt135.
(33) Chaikind, B.; Bessen, J. L.; Thompson, D. B.; Hu, J. H.; Liu, D. R. A Programmable Cas9-Serine Recombinase Fusion Protein That Operates on DNA Sequences in Mammalian Cells. Nucleic Acids Res. 2016, 44 (20), 9758-9770. https://doi.org/10.1093/nar/gkw707.
(34) Nollmann, M.; Byron, O.; Stark, W. M. Behavior of Tn3 Resolvase in Solution and Its Interaction with Res. Biophys. J. 2005, 89 (3), 1920-1931. https://doi.org/10.1529/biophysj.104.058164.
(35) Cremer, T.; Cremer, M. Chromosome Territories. Cold Spring Harb. Perspect. Biol. 2010, 2 (3). https://doi.org/10.1101/cshperspect.a003889.
(36) Gordley, R. M.; Smith, J. D.; Graslund, T.; Barbas, C. F. Evolution of Programmable Zinc Finger-Recombinases with Activity in Human Cells. J. Mol. Biol. 2007, 367 (3), 802-813. https://doi.org/10.1016/j.jmb.2007.01.017.
(37) Gaj, T.; Mercer, A. C.; Gersbach, C. A.; Gordley, R. M.; Barbas, C. F. Structure-Guided Reprogramming of Serine Recombinase DNA Sequence Specificity. Proc. Natl. Acad. Sci. 2011, 108 (2), 498-503. https://doi.org/10.1073/pnas.1014214108.
(38) Gaj, T.; Mercer, A. C.; Sirk, S. J.; Smith, H. L.; Barbas, C. F. A Comprehensive Approach to Zinc-Finger Recombinase Customization Enables Genomic Targeting in Human Cells. Nucleic Acids Res. 2013, 41 (6), 3937-3946. https://doi.org/10.1093/nar/gkt07l.
(39) Hu, J. H.; Miller, S. M.; Geurts, M. H.; Tang, W.; Chen, L.; Sun, N.; Zeina, C. M.; Gao, X.; Rees, H. A.; Lin, Z.; et al. Evolved Cas9 Variants with Broad PAM Compatibility and High DNA Specificity. Nature 2018, 556 (7699), 57-63. https://doi.org/10.1038/nature26155.
(40) Chatterjee, P.; Jakimo, N.; Jacobson, J. M. Minimal PAM Specificity of a Highly Similar SpCas9 Ortholog. Sci. Adv. 2018, 4 (10), eaau0766. https://doi.org/10.1126/sciadv.aau0766.
(41) Nami, F.; Basiri, M.; Satarian, L.; Curtiss, C.; Baharvand, H.; Verfaillie, C. Strategies for In Vivo Genome Editing in Nondividing Cells. Trends Biotechnol. 2018, 36 (8), 770-786. https://doi.org/10.1016/j.tibtech.2018.03.004.
(42) Siuti, P.; Yazbek, J.; Lu, T. K. Synthetic Circuits Integrating Logic and Memory in Living Cells. Nat. Biotechnol. 2013, 31 (5), 448-452. https://doi.org/10.1038/nbt.2510.
(43) Yang, L.; Nielsen, A. A. K.; Fernandez-Rodriguez, J.; McClune, C. J.; Laub, M. T.; Lu, T. K.; Voigt, C. A. Permanent Genetic Memory with >1-Byte Capacity. Nat. Methods 2014, 11 (12), 1261-1266. https://doi.org/10.1038/nmeth.3147.
(44) Weinberg, B. H.; Pham, N. T. H.; Caraballo, L. D.; Lozanoski, T.; Engel, A.; Bhatia, S.; Wong, W. W. Large-Scale Design of Robust Genetic Circuits with Multiple Inputs and Outputs for Mammalian Cells. Nat. Biotechnol. 2017, 35 (5), 453-462. https://doi.org/10.1038/nbt.3805.
(45) Sikorski, R. S.; Hieter, P. A System of Shuttle Vectors and Yeast Host Strains Designed for Efficient Manipulation of DNA in Saccharomyces cerevisiae. Genetics 1989, 122 (1), 19-27.
(46) Ellis, T.; Wang, X.; Collins, J. J. Diversity-Based, Model-Guided Construction of Synthetic Gene Networks with Predicted Functions. Nat. Biotechnol. 2009, 27 (5), 465-471. https://doi.org/10.1038/nbt.1536.s

1. A method of deleting a target sequence from the genome in a eukaryotic cell, wherein the target sequence for deleting is flanked by two Tn3 core recognition sequences, wherein each Tn3 core recognition sequence is flanked by first and second sgRNA target sequences on opposite strands of the genomic DNA and positioned at equal distances around the Tn3 core recognition sequence, the method comprising:
introducing into the cell a nucleotide sequence, the nucleotide sequence encoding a fusion protein consisting of the amino acid sequence of SEQ ID NO: 1;
introducing a first oligonucleotide sequence encoding a first single guide RNA (sgRNA) sequence targeting the first sgRNA target sequence and a second oligonucleotide sequence encoding a second sgRNA sequence targeting the second sgRNA target sequence,
wherein the first and second sgRNA target sequences are spaced to position the 5′ ends of the first and second sgRNAs 22 bp apart, whereby the first and second sgRNAs are on the same helical face of the DNA molecule and the catalytic domains of the hyperactive Tn3 transposon resolvases are positioned to have activity on the Tn3 core recognition sequence;
coexpressing the nucleotide sequence, the first oligonucleotide sequence, and the second oligonucleotide sequence in the eukaryotic cell to generate a transformed eukaryotic cell; and
culturing the transformed eukaryotic cell to remove the region of target sequence from the genome of the cultured eukaryotic cell.

2. The method of claim 1, wherein the first and second sgRNAs target sequences are positioned at equal distances around the Tn3 core recognition sequence.

3. The method of claim 1, wherein the method does not cause off target mutations, nucleotide insertions, and/or nucleotide deletions.

4. The method of claim 1, wherein the portion of the genome is deleted independent of the cell's endogenous DNA repair mechanism.

5. The method of claim 1, wherein the portion of the genome is deleted by triggering non-homologous end joining.

6. A method of inserting an extraneous sequence into a target region of a genome in a eukaryotic cell, wherein the extraneous sequence for inserting is encoded on a donor DNA molecule comprising a Tn3 core recognition sequence and the target region of a genome comprises a Tn3 core recognition sequence, wherein each Tn3 core recognition sequence is flanked by first and second sgRNA target sequences on opposite strands of the DNA and positioned at equal distances around the Tn3 core recognition sequence, the method comprising:
introducing into the cell a nucleotide sequence, the nucleotide sequence encoding a fusion protein consisting of the amino acid sequence of SEQ ID NO: 1;
introducing a first oligonucleotide sequence encoding a first single guide RNA (sgRNA) sequence targeting the first sgRNA target sequence and a second oligonucleotide sequence encoding a second sgRNA sequence targeting the second sgRNA target sequence,
wherein the first and second sgRNA target sequences are spaced to position the 5′ ends of the first and second sgRNAs 22 bp apart, whereby the first and second sgRNAs are on the same helical face of the DNA molecule and the catalytic domains of the hyperactive Tn3 transposon resolvases are positioned to have activity on the Tn3 core recognition sequence;
introducing the donor DNA molecule encoding the extraneous sequence;
coexpressing the nucleotide sequence, the first oligonucleotide sequence, the second oligonucleotide sequence, and the donor DNA molecule in the eukaryotic cell to generate a transformed eukaryotic cell; and
culturing the transformed eukaryotic cell to insert the extraneous sequence into the genome of the cultured eukaryotic cell at the site of the target region.

7. The method of claim 6, wherein the first and second sgRNAs target sequences are positioned at equal distances around the Tn3 core recognition sequence.

8. The method of claim 6, wherein the method does not cause off target mutations, nucleotide insertions, and/or nucleotide deletions.

9. The method of claim 6, wherein the portion of the genome is deleted independent of the cell's endogenous DNA repair mechanism.

10. The method of claim 6, wherein the portion of the genome is deleted by triggering non-homologous end joining.

11. A method of inserting an extraneous sequence into a target region of a genome in a cell, the method comprising:
introducing into the cell a first nucleotide sequence, the first nucleotide sequence encoding a fusion protein consisting of the amino acid sequence of SEQ ID NO: 1;
introducing a first oligonucleotide sequence encoding a first single guide RNA (sgRNA) sequence, a second oligonucleotide sequence encoding a second sgRNA sequence, and a third oligonucleotide encoding a third sgRNA sequence, wherein:
the first sgRNA sequence is complementary to the 5′ end of a target region,
the second sgRNA is complementary to the 3′ end of the target region,
a protospacer adjacent motif is adjacent and proximal to the 5′ end the target region,
a protospacer adjacent motif is adjacent and distal to the 3′ end of the target region,
the distance between the 5′ end of the target region and the 3′ end of the target region is at least 22 bp,
the target region comprises a sequence complementary to a sequence recognized by the catalytic domain of the recombinase of the fusion protein between the 5′ end of the target region and the 3′ end of the target region,
the third sgRNA sequence is complementary to a sequence in the genome of the cell that is at least 20 bp from the 3′ end of the target region, wherein the sequence in the genome of the cell that is at least 20 bp from the 3′ end of the target region comprises a protospacer adjacent motif distal to the sgRNA sequence;
introducing a second nucleotide sequence encoding the extraneous sequence and a recognition site sequence for the fusion protein, wherein:
the recognition site is proximal to the extraneous sequence, and
the recognition sequence comprises a sequence complementary to the region of the genome comprising the target region and at least 21 bp from the 3′ end of the target region;
coexpressing the first nucleotide sequence, the first oligonucleotide sequence, the second oligonucleotide sequence, the third oligonucleotide sequence, and the second nucleotide sequence in the eukaryotic cell to generate a transformed eukaryotic cell; and
culturing the transformed eukaryotic cell to insert the extraneous sequence into the genome of the cultured eukaryotic cell at the site of the target region.

12. The method of claim 11, wherein the distance between the 5′ end of the target region and the 3′ end of the target region is 22 bp, 30 bp, 31 bp, or 44 bp.

13. The method of claim 11, wherein the third sgRNA sequence is complementary to a sequence in the genome of the cell that is 20 bp from the 3′ end of the target region.

14. The method of claim 11, wherein the third sgRNA sequence is complementary to a sequence in the genome of the cell that is 21 bp from the 3′ end of the target region.

15. The method of claim 11, wherein the first and second sgRNAs target sequences are positioned at equal distances around the Tn3 core recognition sequence.

16. The method of claim 11, wherein the method does not cause off target mutations, nucleotide insertions, and/or nucleotide deletions.

17. The method of claim 11, wherein the portion of the genome is deleted independent of the cell's endogenous DNA repair mechanism.

18. The method of claim 11, wherein the portion of the genome is deleted by triggering non-homologous end joining.
